SEQUENCE LISTING (63 sequences)

SEQ ID NO: 1 - DNA, 1250 bp, Homo sapiens
cgccgccatagctgcatggccccgggacctccctgtcgtacctgagaggagggcctggcccgtgaactgcccgtacacggaggcagcatggggaaaggcattgaagggcgggaccccggagccgacttgctgcagatccaaaaaggcggagctagataaagaggaaggggtggagctagaactggacacctcgggggtttcctgctttatggcgaagggtgagtgaggatctgccggagggagggagacaacaaggagagaggggtgtgagatggcggggagggaacacgcacaagccaagccgtgggatgtggagggtgcgggcggggcgcggggcaggctcagctgccctcagagtctgggctggggagagccggggcccacgaggcgtggcaaggcggggagagagaagctagacctccctgctgcgcttctgcagcgaaaatggagagacccggaaggcgtgtgtgcgcccgcctctcccacaggagggagcgcggagacccgggagcggcctaggaccggaggcgcgacccctcggcccaccttgaggcccggcctaggaccggaggcgcgacccctgggcccaccttgaggcccggcctaggactggaggcgcgacccctcggcccaccttgcgggagccgcctaggaccggaggcgcgacccctcggcccagcttgaggcccggcctaggaccggaggcgcgacccctcggcccaccttgcgggagccgcctaggaccggaggcgcgacccctcggcccaccttgaggcccggcctaggaccggaggcgcgacccctgggcccaccttgaggcccggcctaggactggaggcgcgacccctcggcccaccttacgggagccgcctagggccggaggcgcgacccctcggcccagcttgaggcccggcctaggaccggaggcgcgacccctcggcccaccttgaggcccggcctaggactggaggcgcgacccctcggcccaccttgaggcccggcctaggaccggaggcgcgacccctgggcccaccttgaggcccggcctaggactggaggcgcgacccctcggcccaccttgaggcccggcctaggaccggaggcgcgacccctgggcccacctggcggcccggccggcacagcccgcctctcctctccaggccagggccccacaccttccgcctgacagccagccaagctcttcagtcccccgccctccacctgccagggaggctccgggcgttgtacct

SEQ ID NO: 2 - DNA, 609 bp, Homo sapiens
agactcgctcaattccctcattattaagctgatccaggcagatattttgaaaaacaagctctccaagcagatggtggacgttaaggaaaattaccagagcaccctgcccaaagctgaggctccccgagagccggagcggggagggcccgccaagtcagcattccagccggtgattgcaatggacaccgaactgctgcgacaacagagacgctacaactcaccgcgggtcctgctgagcgacagcacccccttggagcccccgcccttgtatctcatggaggattacgtgggcagccccgtggtggcgaacagaacatcacggcggaaacggtacgcggagcataagagtcaccgaggggagtactcggtatgtgacagtgagagtctgtgggtgaccgacaagtcatcggccatcgacattcggggacaccaggtcacggtgctgggggagatcaaaacgggcaactctcccgtcaaacaatatttttatgaaacgcgatgtaaggaagccaggccggtcaaaaacggttgcaggggtattgatgataaacactggaactctcagtgcaaaacatcccaaacctacgtccgagcactgacttcagagaa

SEQ ID NO: 3 - DNA, 552 bp, Homo sapiens
  misc_feature (411)..(411): n is a, c, g, or t
aaaaagtccacagtttaacagttcctccccaacctgtaaccccgccttgaacttctggactagcccctcgattgttgtagatgccaagcggacctcgcgccgctctgcgttgggccagcccctcacagctggtttcttaccacgtattgcgcaagcggaatctatgcctgttacccacactccctgcgcccccgcaccccgctcctgtgcgcaagtcggaatataaaaccgcggaggagtgagctcttggggtgtccagttggttgccgcggcagtctctccgagcagcgcatttgtcttctaggctgcttggttcgtgcctccgagaaaggtaagtctttctttcgcttttttaggggtacttgaaaacaacaagtgtcagacaaagcagcagatgctgttgcgcagtanaagtttatgggcgagttgtccctgaaactggaaccaggtctttcttggcgcgattacgcaagaaccacccgcagccctgcgggctcctggcaggtcctgcaactgcactttggatagtcccgttgggaagctagcactttt

SEQ ID NO: 4 - DNA, 1209 bp, Homo sapiens
tatttttttagagaagttgacttgctatgtggactaggcaggactggaagtcctgggctcaagtgatgctcccgcctcagcctcctaagtagcttggactacagcttcccgccacctcccccatcttgctttttagtttaaagcagggtcagcacatcacatgaagtcatctcctttttggggatatcccacatgtccagaactaccagacggtagtggggtggccggctaggctgtggggagcacggagatttatttgcaaaggaggacctggacaaatgtgcccccacatcctctcaggcgaggagaatggacgagagtgagaggccgacccgtgttcccgtgttactgtgtacggagtagtgggtccgagggacctaggtgtggacagggacaggcaaggcgacagcgaggagaaacgaaaatcacatcggtggcggttgctctgcacacaactcgctcgctaccgcacgctccacgctctgcactacgccgatccggggacaggagcaggaggctgtggctgcactcagacttcgggacaggccgagctgaaaaccgtgagaggggtggggtggaggcgaccgaaacgccaaggctgggttcccggaacgcgcggggactagggtggaaggcaacttcggggaaactgggaaaggcgaccgggacctcggggacgccccgtaccccgggcgtaaactcactcccgcgttagcgggcgccaaagcggggagggggtggtcccgtggtccgcacccaggggagctcagtggactgtgcgccttgcctttctgctgcgcaaagcccagtccaggtcatcacctcgggcggggcggactcggctgggcggactcagcggggcgggcgcaggcgcagggcgggtcctttgcgtccggccctctttcccctgaccataaaagcagccgctggctgctgggccctaccaagccttccacgtgcgccttatagcctctcaacttcttgcttgggatctccaacctcaccgcggctcgaaatggaccccaactgctcctgcgccactggtaagggatgctaggtttctggtccttaggatacctatttccccgccacaggatagatgtccctaggagtagaggtgttttttgagttctagctaagtggagtcatttatttcattgatctagtgcttttccactcagcgccttcatcatccctagaacattccta

SEQ ID NO: 5 - DNA, 2799 bp, Homo sapiens
ctcattcaagcagtatttattaggggccagctttgtggccggcaccgtggcgggctctggggctacaaaaggtgaataatacttgggctctgcctctgagggccttacacgttagggaggagtgggtcaactgccacaaacgtcgctaggaaattaaaaggaaaattttacaaagtggcagttcttgtctgtcttcccctccagatggcccgtgtgttgttttcgggccggggctatttctcatttatttcgcacccccggttcttagtgccctgtaggtgctaaatcagcatttgtttcatgagtgctttttctgggggcaaccagacccctgcagaagtgtacctgtgttgtgccagaggttctgatgataggcttataggcggtagtttcctcagtgtccgtgggtcgcccccggtcccgggttggatgccccgcggtccagcaccgaacctttcggggtgcagagttgcagagccgcggagggcccgggccgtgcgcagccgaagggaggcctgcagcgccccctctggatgcagcgggcaccggccggccgccccgctcacccgctcgcaccccacgtttgttcaccagtatttcagtttacggtcagaaaatgaacacagacacttcgtgatactctacacttttcaaaggcgtaagggatgccttttaaaggattatggattagaaaaattcctccctctttcttgtgcctctgggcccttgcattgtgattctatcttacgtaaataaagggggctttgctctcctaattgtgcccactgttctgtgcagcgcggaccggcgcatgcagcgagcggggctgcgagggcgctgctgtggccaggcgtctggcatgctgaccacgtcgcgctgctgtaaaggaaacctgccccgcgcagcggcggtggctggagcgggagaaaccggactttgtgcaactttggccatagtggccatcccatgaatctgtttactagcttggtggtgggtccaacagagcttgttgctccctagccgcttgctcgtgcccttggtggttaccggtagttaagcttagggcgcatagggccctcgtggctcgccacctctcacggttcagtacccacgcttcgaacgagggatgggagcaggcgccacggccggcaccccagagccctgctgccccttagttcgagcggccatcctcctgtggggcttgtgggcagcctgtggggtttgtgggcggcctgtggggtttgtgggtggtctaaggaaagagttggggcactcaggggtctgctgtttttgcccgtggccttaactcatcaggggagggtttctgcagcagaatctcgggctcagggttggcggttaacgagggagcagcggggtcttggggagggggctcgacacccctgaaggtgccccctaaaggagccactgttagaggggcaccccatctttgtggccatggcggtggtagagcggctgggaggggctctgcggcgagcaagggagcaggcggtaggggttctgcggcgatgggcgggctaggggcggggcgcgggtgggctctaaaagtcggtgcccactcgctccgcgctgccgcggcaaccagcacaccccggcacctcctctgcggcagctgcgcctcgcaagcgcagtgccgcagcgcacgccggagtggctgtagctgcccggcgcggcgccgccctgcgcgggctgtgggctgcgggctgcgcccccgctgctggccagctctgcacggctgcgggctctgcggcgcccggtgctctgcaacgctgcggcgggcggcatgggataacgcggccatggtgcgccgagatcgcctccgcaggtgagtgtgcggtgggaacgagggggtgtggctggcggccctgggactagggcgcaggcgagcggaggactgtgtgcccgtgtccgagctggggctgcctctgggcgaaaactctaccgacaggcggcacgcattccgcgcccgctctgcctacttgaggagggggtgtcactcctgcccgcaatggaatgttcagaacgcgggacctccttgggttaggatttctagaccccgggatcgtcgtggtgagatttaggatttctggaccccagcgtcatcttgatatgacttaggatccataatgaccctggtctcaccctgatgcgaattgggatttttagatcctggcatcaccctggtgcgatttaggatttttatactcagtcattgctgcagcatgatttaggatttctaacccccagcatcgccctggtttgatttaggatatttagactccggcttccctctggtgcgattcaggattcttagactccgccgttgccgtggcgcgatttaggatttatagatcccggcaaagccctggtgcgatgtaggatttttagaaccccagcatcgctctggtgcgacttaaaggataggccccagcatcgccctggtgcgatgtaggatttttagaaccccggtgtctccgtggcgcaccttaggatttcaagaacgggataatcgcagtgccgagatcgccgcggtgcagcttaggatttcaagacccaggtatcacggtggcgggagtcaccgcagtgactagaactcgcagtgcccgtcagccgccttaagtatttttcagatttcagtaacaagcgcgagtgagaacggcgatgtgaccaaactgtcatgttgcgcagggattgttcaccttggtttcgcgggttttcaaagtggttcgtctcgcggcgacgccatcaggtgggcggcaggttgggtggtattattacggg

SEQ ID NO: 6 - DNA, 661 bp, Homo sapiens
ccgaacgctatgaggacatggcagccttcatgaaaggcgccgtggagaagggcgaggagctctcctgcgaagagcgaaacctgctctcagtagcctataagaacgtggtgggcggccagagggctgcctggagggtgctgtccagtattgagcagaaaagcaacgaggagggctcggaggagaaggggcccgaggtgcgtgagtaccgggagaaggtggagactgagctccagggcgtgtgcgacaccgtgctgggcctgctggacagccacctcatcaaggaggccggggacgccgagagccgggtcttctacctgaagatgaagggtgactactaccgctacctggccgaggtggccaccggtgacgacaagaagcgcatcattgactcagcccggtcagcctaccaggaggccatggacatcagcaagaaggagatgccgcccaccaaccccatccgcctgggcctggccctgaacttttccgtcttccactacgagatcgccaacagccccgaggaggccatctctctggccaagaccactttcgacgaggccatggctgatctgcacaccctcagcgaggactcctacaaagacagcaccctcatcatgcagctgctgcgagacaacctgacactgtggacggccgacaacgccgg

SEQ ID NO: 7 - DNA, 1478 bp, Homo sapiens
tccccattccgccctgaaagttggatgcggagactaacagaagtcgcattatcagctgtcccgatctaggaaatttttaggaccccacgtttttaaatacttttaagagtatgtctgatacagtctgtaatactaaagcatcaaaataatcatattttccataagagacgaaagtgcaaacagttactgtctagtcccattattacttggaacagactttttcttcttttccttgttctgtttttttcctggcccgttggcgaggttagagcgccaggttgtaagaatcgggtctgtggacctcataccagataggcgcgaacgcctctggcagcggcgtccagggggtccggcggcactcgcggtggggctgcctgggttgcgggtgacgatctgcggggtcccgcacccggccccgcggagcccggacccgcacgtaggcggcgcggcaaaggcacaccctcctcgcggccgcgaacccagcgccgtcctcgcagcgcggcaatgcacggccaccgctgcccccagcccgcccgccgcagccgcgagcacccaaacacctaccctgcggggcgacgacccccggagctcaggcgaggccgctcgggcgtgccacctccgcggccatgacggcgacccggggaagcgccccgcgcgccaaggccccgcgctgctgagctgtgagcacggctgccccgtccgtccgtccgtccagcacccgcccggagagtgaggccagagcacgccccagccgtgtctaaatcaaggctcggggcggtaccgacgggctgaatgacaaatggcagatgccgtgggctttgccgcccgcggcagccaagaggatggctgcgccgaggaggccgcgcgcaggcggggctcgggagccggaacggcgcggccgcgaggagggcgctggggcccctggcgggggcgtcacgtggcaggaggaggccccgccggggagctgggggtcggcggccgaggcggggggagctgagcggcacccacacgtcctgcgggccgggtcaccggtgggggcaaagccaaggtcgcccaggtacagcgctggcgcaggtagacccgagccggcctggggtctgcagcggggcctgctagccgaagtctccgccaggatgggccgccagagcccaatcacacatgagaaacgcgacagatgctgggacgctgcaataggccaaactaacttacctcctgtgccagcagcgcccaagtgcagctgcccaaacgtgagcactgaccgtgagccaggcactgtcctaagcactttgcaggtaaatacgtgtaatcctcacagcaacactgggagaaatacccgtctcacagctgaagaaacgaagacgcagaaagggtagacagaagtaattttctaattacggtatcacactacgtctgcttcatataatttcaaatttttcatgttactggaatttaagaaaaataaactgaagggaatctct

SEQ ID NO: 8 - DNA, 448 bp, Homo sapiens
tgccccctcctctcctggggtgctgagacgagggactcccctcctctagaggaagcaggagacagggccacagcaccatgcaggggaccaggggctgcagccagccctatcctggctgtgtcctgggctcgcccgcagcagctgctggcacctggacggcggcgccaggctcacctctatagtggggtcgtattcgtccacaaaatggttctggatcagctggatggtcagcgcactcttgcccacaccgccggcgcccaccaccaccagcttatattccgtcatcgctcctcaggggcctgcggcccggggtcctcctacagggtctcctgccccacctgccaaggagggccctgctcagccaggcccaggcccagccccaggccccacagggcagctgctggcagggccatctgaagggcaaacccacagcggtccctgggcccca

SEQ ID NO: 9 - DNA, 500 bp, Homo sapiens
tgaaaggagtcatcatctaatggaattgcatggaatcatcataaaatggaatcgaatggaatcaacatcaaatggaatcaaatggaatcattgaacggaattgaatggaatcgtcatcgaatgaattgactgcaatcatccaatggtcgcgaatggaatcatcttcaaatggaatggaatggaatcatcgcatagaatcgaatggaattatcatcgaatggaatcgaatggaatcaacatccaacggaaaaaaacggaattatcgaatggaatcgaagagaatcatcgaatggacccgaatggaatcatctaatggaatggaatggaataatccatggactcgaatgcaatcatcatcgaatggaatcgaatggaatcatcgaatggactcgaatggaataatcattgaacggcatcgaatggaatcatcatcggatggaaatgaatggaatcatcatcgaatggaatcgaatagaattatggaatgaaatccagtgtga

SEQ ID NO: 10 - DNA, 850 bp, Homo sapiens
tttccgagtccccgtggggagccggggaccgtcccgcccccgtcccccgggtgccggggagcggtccctctgccgcgatcctttctggcgagtccccgtgcggagtcggagagcgctccctgagcgcgcgtgcggcccgagaggtcgcgcctggccggccttcggtccctcgtgtgtcccggtcgtaggaggggccggccgaaaatgcttccggctcccgctctggagacacgggccggccccctgcgtgtggcacgggcggccgggagggcgtccccggcccggcgctgctcccgcgtgtgtcctggggttgaccagagggccccgggcgctccgtgtgtggctgcgatggtggcgtttttggggacaggtgtccgtgtcgcgcgtcgcctgggccggcggcgtggtcggtgacgcgacctcccggccccggggaggtatatctttcgctccgagtcggcattttgggccgccgggttattgctgacacgctgtcctctggcgacctgtcgctggagaggttgggcctccggatgcgcgcggggctctggcctaccggtgacccggctagccggccgcgctcctgcttgagccgcctgccggggcccgcgggtcgctgttctctcgcgcgtccgagcgtcccgactcccggtgccggcccgggtccggtctctggccacccgggggcggcgggaaggcggcgagggccaccgtgccccgtgcgctctccgctgcgggcgcccggggcgcgcaaccccaccccgctggctccgtgccgtgcgtgtcaggcgttctcgtctccgcggggcttgtccgccgccccttccccggagtgggggttggccggagt

SEQ ID NO: 11 - DNA, 743 bp, Homo sapiens
ttggagagggggcgtcatcacctcacccaaaggttaaataggggttgagatatgatgctcaggagaagcgctttctttcgcgagcaccctgaaccagaccatgacccagaccctcaagtacgcctccagagtgttccatcgcgtccgctgggcgccttgggcgcctccctaggctaccgagagtaccactcagcacgccggagcttggcagacatcccaggcccctctacgcccagctttctggccgaacttttctgcaagggggggctgtcgaggctacacgagctgcaggtaggaagggacgcctttcccgagacagagtgctggggaaactggttttgacagcgtcagaaaggactgactagtgcagagcaaatgtgggacagccagagagaacggatgcccatgaaataaggaaaaggcgagttgaggctgggggcggtgtggctacactcgggcagagcccgtcccgactcttagcagagggcgctgcgaaagcgccttctcgctgtcctgaggtgtggagatcctgcagataaagtacaagtgcgcgggagggggaggccggagtgggcagtaccctccgcctgcttgcggcttaaagctacatgggttccttttcattcactgaggactcgtcctgagatggacagtccagacatggaacttttagagatttctcctcgaccgagatgatcagagaggtcctgaatgtctgccttgcacaaagttccggttttgcc

SEQ ID NO: 12 - DNA, 2038 bp, Homo sapiens
tgggctcaagtgatcctcccatctcggcctcccaaaatgctgggattacaggtgggagccgcgcccaggtggatttttgtctgactctgttcattcctgtgtccccagtacctggaaggacgccaagcacacagtaggcgcttaaaaaacattgagccacatgttgagaaaagaacggcaccattgtggctgcaagtgggacttgggccgcgcgggggagctcgcgcacctcgggccggggcaagagctcagtggaacccgcccgaggaagaacccgtggcgcaggattttcccaggccttctgaggaccaggggcgtcccccgtcccaccctgtgactttgctcaggccgttccggggcgggaattcagaactcctcagccccccaagaaaaaaatatccccgtggaaattccttgggaatgaccgaggcgggggaaatatgcgtctctggatggccagtgactcgcagcccccttccccgataggaagggcctgcgcgtccggggacccttcgcttccccttctgctgcgcgacctccctggcccctcggagatctccatggcgacgccgcgcgcgccccacaacaggaaagccttaggcggcgcggcttggtgctcggagacttaagagtacccagcctcgacgtggtggatgtcgagtcttggggtcacacgcacaggcggtggccaagcaaacacccgctcatatttagtgcatgagcctgggttcgagttgccggagcctcgcgcgtagggcaggggttcgagcgccccttctccctgcctcgcctctgcgcctgggggctgctgcctcagtttcccagcgacaggcagggatttcgagcgtccccctcccctccctcgtcaagatccaagctagctgcctcagtttccccgcggagcctgggacgccagcggaggggctcggcgcgtagggatcacgcagcttccttcctttttctgggagctgtaaagacgcctccgcggccaaggccgaaaggggaagcgaggaggccgccggggtgagtgccctcgggtgtagagagaggacgccgatttccccggacgtggtgagaccgcgcttcgtcactcccacggttagcggtcgccgggaggtgcctggctctgctctggccgcttctcgagaaatgcccgtgtcagctaggtgtggacgtgacctagggggaggggcatccctcagtggagggagcccggggaggattcctgggcccccacccaggcagggggctcatccactcgattaaagaggcctgcgtaagctggagagggaggacttgagttcggaccccctcgcagcctggagtctcagtttaccgctttgtgaaatggacacaataacagtctccactctccggggaagttggcagtatttaaaagtacttaataaaccgcttagcgcggtgtagaccgtgattcaagcttagcctggccgggaaacgggaggcgtggaggccgggagcagcccccggggtcatcgccctgccaccgccgcccgattgctttagcttggaaattccggagctgaagcggccagcgagggaggatgaccctctcggcccgggcaccctgtcagtccggaaataactgcagcatttgttccggaggggaaggcgcgaggtttccgggaaagcagcaccgccccttggcccccaggtggctagcgctataaaggatcacgcgccccagtcgacgctgagctcctctgctactcagagttgcaacctcagcctcgctatggctcccagcagcccccggcccgcgctgcccgcactcctggtcctgctcggggctctgttcccaggtgagtcggggtggggattgccgtcgggccagttctccgaagccccgggaggaccggctcccgggtcaggtcatgcatgcttaggtagctgtttatgggaaggaggggctagagacagcgattgaaaggcaacagccagtaggttcgaatccagaccctgcatacctccacgtgtggccttgggctatagattgcagctttaaaaaagggtaggg

SEQ ID NO: 13 - DNA, 77 bp, Homo Sapien
cgggacctccctgtcgtacctgagaggagggcctggcccgtgaactgcccgtacacggaggcagcatggggaaaggc

SEQ ID NO: 14 - DNA, 74 bp, Homo Sapien
ccccgcccttgtatctcatggaggattacgtgggcagccccgtggtggcgaacagaacatcacggcggaaacgg

SEQ ID NO: 15 - DNA, 86 bp, Homo Sapien
gcgcaagcggaatctatgcctgttacccacactccctgcgcccccgcaccccgctcctgtgcgcaagtcggaatataaaaccgcgg

SEQ ID NO: 16 - DNA, 80 bp, Homo Sapien
cgtgttcccgtgttactgtgtacggagtagtgggtccgagggacctaggtgtggacagggacaggcaaggcgacagcgag

SEQ ID NO: 17 - DNA, 87 bp, Homo sapiens
cggcgcccggtgctctgcaacgctgcggcgggcggcatgggataacgcggccatggtgcgccgagatcgcctccgcaggtgagtgtg

SEQ ID NO: 18 - DNA, 81 bp, Homo Sapien
gaggagggctcggaggagaaggggcccgaggtgcgtgagtaccgggagaaggtggagactgagctccagggcgtgtgcgac

SEQ ID NO: 19 - DNA, 77 bp, Homo Sapien
accgacgggctgaatgacaaatggcagatgccgtgggctttgccgcccgcggcagccaagaggatggctgcgccgag

SEQ ID NO: 20 - DNA, 96 bp, Homo Sapien
cgtccacaaaatggttctggatcagctggatggtcagcgcactcttgcccacaccgccggcgcccaccaccaccagcttatattccgtcatcgctc

SEQ ID NO: 21 - DNA, 80 bp, Homo Sapien
tcgaatggaatcaacatccaacggaaaaaaacggaattatcgaatggaatcgaagagaatcatcgaatggacccgaatgg

SEQ ID NO: 22 - DNA, 75 bp, Homo sapiens
cgctctggagacacgggccggccccctgcgtgtggcacgggcggccgggagggcgtccccggcccggcgctgctc

SEQ ID NO: 23 - DNA, 78 bp, Homo sapiens
gggacagccagagagaacggatgcccatgaaataaggaaaaggcgagttgaggctgggggcggtgtggctacactcgg

SEQ ID NO: 24 - DNA, 80 bp, Homo sapiens
ggccagcgagggaggatgaccctctcggcccgggcaccctgtcagtccggaaataactgcagcatttgttccggagggga

SEQ ID NO: 25 - DNA, 23 bp, Artificial Sequence
  PAX8 Forward Primer
cgggatttttttgtcgtatttga

SEQ ID NO: 26 - DNA, 22 bp, Artificial Sequence
  PAX8 Reverse Primer
acctttccccatactacctccg

SEQ ID NO: 27 - DNA, 28 bp, Artificial Sequence
  PAX8 Oligonucleotide Probe [5′ 6FAM and 3′ BHQ1]
acgaacaattcacgaaccaaaccctcct

SEQ ID NO: 28 - DNA, 27 bp, Artificial Sequence
  NTF3 Forward Primer
tttcgtttttgtattttatggaggatt

SEQ ID NO: 29 - DNA, 20 bp, Artificial Sequence
  NTF3 Reverse Primer
ccgtttccgccgtaatattc

SEQ ID NO: 30 - DNA, 23 bp, Artificial Sequence
  NTF3 Oligonucleotide Probe [5′ 6FAM and 3′ BHQ1]
tcgccaccacgaaactacccacg

SEQ ID NO: 31 - DNA, 23 bp, Artificial Sequence
  DIRAS3 Forward Primer
gcgtaagcggaatttatgtttgt

SEQ ID NO: 32 - DNA, 22 bp, Artificial Sequence
  DIRAS3 Reverse Primer
ccgcgattttatattccgactt

SEQ ID NO: 33 - DNA, 29 bp, Artificial Sequence
  DIRAS3 Oligonucleotide Probe [5′ 6FAM and 3′ BHQ1]
cgcacaaaaacgaaatacgaaaacgcaaa

SEQ ID NO: 34 - DNA, 24 bp, Artificial Sequence
  MT1A Forward Primer
cgtgttttcgtgttattgtgtacg

SEQ ID NO: 35 - DNA, 22 bp, Artificial Sequence
  MT1A Reverse Primer
ctcgctatcgccttacctatcc

SEQ ID NO: 36 - DNA, 27 bp, Artificial Sequence
  MT1A Oligonucleotide Probe [5′ 6FAM and 3′ BHQ1]
tccacacctaaatccctcgaacccact

SEQ ID NO: 37 - DNA, 20 bp, Artificial Sequence
  MEST Forward Primer
cggcgttcggtgttttgtaa

SEQ ID NO: 38 - DNA, 25 bp, Artificial Sequence
  MEST Reverse Primer
cacactcacctacgaaaacgatctc

SEQ ID NO: 39 - DNA, 28 bp, Artificial Sequence
  MEST Oligonucleotide Probe [5′ 6FAM and 3′ BHQ1]
acgcaccataaccgcgttatcccatacc

SEQ ID NO: 40 - DNA, 20 bp, Artificial Sequence
  SFN Forward Primer
gaggagggttcggaggagaa

SEQ ID NO: 41 - DNA, 20 bp, Artificial Sequence
  SFN Reverse Primer
atcgcacacgccctaaaact

SEQ ID NO: 42 - DNA, 25 bp, Artificial Sequence
  SFN Oligonucleotide Probe [5′ 6FAM and 3′ BHQ1]
tctcccgatactcacgcacctcgaa

SEQ ID NO: 43 - DNA, 23 bp, Artificial Sequence
  PLAGL1 Forward Primer
atcgacgggttgaatgataaatg

SEQ ID NO: 44 - DNA, 20 bp, Artificial Sequence
  PLAGL1 Reverse Primer
ctcgacgcaaccatcctctt

SEQ ID NO: 45 - DNA, 25 bp, Artificial Sequence
  PLAGL1 Oligonucleotide Probe [5′ 6FAM and 3′ BHQ1]
actaccgcgaacgacaaaacccacg

SEQ ID NO: 46 - DNA, 24 bp, Artificial Sequence
  HRAS Forward Primer
gagcgatgacggaatataagttgg

SEQ ID NO: 47 - DNA, 29 bp, Artificial Sequence
  HRAS Reverse Primer
cgtccacaaaataattctaaatcaactaa

SEQ ID NO: 48 - DNA, 23 bp, Artificial Sequence
  HRAS Oligonucleotide Probe [5′ 6FAM and 3′ BHQ1]
cactcttacccacaccgccgacg

SEQ ID NO: 49 - DNA, 28 bp, Artificial Sequence
  SAT2CHRM1 Forward Primer
tcgaatggaattaatatttaacggaaaa

SEQ ID NO: 50 - DNA, 25 bp, Artificial Sequence
  SAT2CHRM1 Reverse Primer
ccattcgaatccattcgataattct

SEQ ID NO: 51 - DNA, 24 bp, Artificial Sequence
  SAT2CHRM1 Oligonucleotide Probe [5′ 6FAM and 3′ MGBNFQ]
cgattccattcgataattccgttt

SEQ ID NO: 52 - DNA, 20 bp, Artificial Sequence
  RNR1 Forward Primer
cgttttggagatacgggtcg

SEQ ID NO: 53 - DNA, 18 bp, Artificial Sequence
  RNR1 Reverse Primer
aaacaacgccgaaccgaa

SEQ ID NO: 54 - DNA, 21 bp, Artificial Sequence
  RNR1 Oligonucleotide Probe [5′ 6FAM and 3′ BHQ1]
accgcccgtaccacacgcaaa

SEQ ID NO: 55 - DNA, 26 bp, Artificial Sequence
  CYP27B1 Forward Primer
gggatagttagagagaacggatgttt

SEQ ID NO: 56 - DNA, 20 bp, Artificial Sequence
  CYP27B1 Reverse Primer
ccgaatataaccacaccgcc

SEQ ID NO: 57 - DNA, 30 bp, Artificial Sequence
  CYP27B1 Oligonucleotide Probe [5′ 6FAM and 3′ BHQ1]
ccaacctcaactcgccttttccttatttca

SEQ ID NO: 58 - DNA, 21 bp, Artificial Sequence
  ICAM1 Forward Primer
ggttagcgagggaggatgatt

SEQ ID NO: 59 - DNA, 24 bp, Artificial Sequence
  ICAM1 Reverse Primer
tcccctccgaaacaaatactacaa

SEQ ID NO: 60 - DNA, 30 bp, Artificial Sequence
  ICAM1 Oligonucleotide Probe [5′ 6FAM and 3′ BHQ1]
ttccgaactaacaaaatacccgaaccgaaa

SEQ ID NO: 61 - DNA, 2799 bp, Artificial Sequence
  HB-493 (MEST) bisulfite treated/converted CpG island sequence
tttatttaagtagtatttattaggggttagttttgtggtcggtatcgtggcgggttttggggttataaaaggtgaataatatttgggttttgtttttgagggttttatacgttagggaggagtgggttaattgttataaacgtcgttaggaaattaaaaggaaaattttataaagtggtagtttttgtttgtttttttttttagatggttcgtgtgttgttttcgggtcggggttattttttatttatttcgtattttcggtttttagtgttttgtaggtgttaaattagtatttgttttatgagtgttttttttgggggtaattagatttttgtagaagtgtatttgtgttgtgttagaggttttgatgataggtttataggcggtagtttttttagtgttcgtgggtcgttttcggtttcgggttggatgtttcgcggtttagtatcgaatttttcggggtgtagagttgtagagtcgcggagggttcgggtcgtgcgtagtcgaagggaggtttgtagcgttttttttggatgtagcgggtatcggtcggtcgtttcgtttattcgttcgtattttacgtttgtttattagtattttagtttacggttagaaaatgaatatagatatttcgtgatattttatattttttaaaggcgtaagggatgttttttaaaggattatggattagaaaaattttttttttttttttgtgtttttgggtttttgtattgtgattttattttacgtaaataaagggggttttgtttttttaattgtgtttattgttttgtgtagcgcggatcggcgtatgtagcgagcggggttgcgagggcgttgttgtggttaggcgtttggtatgttgattacgtcgcgttgttgtaaaggaaatttgtttcgcgtagcggcggtggttggagcgggagaaatcggattttgtgtaattttggttatagtggttattttatgaatttgtttattagtttggtggtgggtttaatagagtttgttgttttttagtcgtttgttcgtgtttttggtggttatcggtagttaagtttagggcgtatagggttttcgtggttcgttattttttacggtttagtatttacgtttcgaacgagggatgggagtaggcgttacggtcggtattttagagttttgttgttttttagttcgagcggttatttttttgtggggtttgtgggtagtttgtggggtttgtgggcggtttgtggggtttgtgggtggtttaaggaaagagttggggtatttaggggtttgttgtttttgttcgtggttttaatttattaggggagggtttttgtagtagaatttcgggtttagggttggcggttaacgagggagtagcggggttttggggagggggttcgatatttttgaaggtgttttttaaaggagttattgttagaggggtattttatttttgtggttatggcggtggtagagcggttgggaggggttttgcggcgagtaagggagtaggcggtaggggttttgcggcgatgggcgggttaggggcggggcgcgggtgggttttaaaagtcggtgtttattcgtttcgcgttgtcgcggtaattagtatatttcggtattttttttgcggtagttgcgtttcgtaagcgtagtgtcgtagcgtacgtcggagtggttgtagttgttcggcgcggcgtcgttttgcgcgggttgtgggttgcgggttgcgttttcgttgttggttagttttgtacggttgcgggttttgcggcgttcggtgttttgtaacgttgcggcgggcggtatgggataacgcggttatggtgcgtcgagatcgttttcgtaggtgagtgtgcggtgggaacgagggggtgtggttggcggttttgggattagggcgtaggcgagcggaggattgtgtgttcgtgttcgagttggggttgtttttgggcgaaaattttatcgataggcggtacgtatttcgcgttcgttttgtttatttgaggagggggtgttatttttgttcgtaatggaatgtttagaacgcgggatttttttgggttaggatttttagatttcgggatcgtcgtggtgagatttaggatttttggattttagcgttattttgatatgatttaggatttataatgattttggttttattttgatgcgaattgggatttttagattttggtattattttggtgcgatttaggatttttatatttagttattgttgtagtatgatttaggatttttaatttttagtatcgttttggtttgatttaggatatttagatttcggtttttttttggtgcgatttaggatttttagatttcgtcgttgtcgtggcgcgatttaggatttatagatttcggtaaagttttggtgcgatgtaggatttttagaattttagtatcgttttggtgcgatttaaaggataggttttagtatcgttttggtgcgatgtaggatttttagaatttcggtgttttcgtggcgtattttaggattttaagaacgggataatcgtagtgtcgagatcgtcgcggtgtagtttaggattttaagatttaggtattacggtggcgggagttatcgtagtgattagaattcgtagtgttcgttagtcgttttaagtattttttagattttagtaataagcgcgagtgagaacggcgatgtgattaaattgttatgttgcgtagggattgtttattttggtttcgcgggtttttaaagtggttcgtttcgcggcgacgttattaggtgggcggtaggttgggtggtattattacggg

SEQ ID NO: 62 - DNA, 1352 bp, Homo sapiens
gatcatcatcgaatggacccgaatggaatcaatcatccaacggaagctaatggaatcaacatcgaatgaatcgaatggaaacaccatcgaattgaaacgaatggaattctcatgaaattgaaatggatggactcgtcatcgaatggattcgaatggaatcatcgaataaaattgattgaaatcatcatcaagtggaatcgaatggtatcattgaatggaatcgaatggaatcatcagatggaaatgaattgaatcgtcatagaatggaatcgaatggattcattgaatggaatcagatggaatcatcgaatggactggaatggaatcattgaatggactcgaaaggaatcatcatcaaatggaaccgaatgaatcctcattgaatggaaatgaaaggggtcatcatctaatggaatcgcatggaatcatcatcaaatggaatcgaatggaatcatcatcaaatggcaatctaatggaatcattgaacagaattgaatggaatcgtcatcgaatgaattgaatgcaatcatcgaatggtctcgaatggaatcatcttctaatggaaaggaatggaatcatcgcatagaatcgaatggaattatcatcgaatggaatcgaatggtatcaaacggaaaaaaacggaattatcgaatggaatcgaagagaatcttcgaacggacccgaatggaatcatctaatggaatggaatggaataatccactggactcgaatgcaatcatcatcgaatggaatggaatggaatcatcgaatggactcgaatggatggaacattgaatcgaatggaatcatcaatcggatggaaacgaatggaatcatcatcgaatggaaatgaaaggagtcatcatctaatggaattgcatggaatcatcataaaatggaatcgaatggaatcaacatcaaatggaatcaaatggaatcattgaacggaattgaatggaatcgtcatcgaatgaattgactgcaatcatccaatggtcgcgaatggaatcatcttcaaatggaatggaatggaatcatcgcatagaatcgaatggaattatcatcgaatggaatcgaatggaatcaacatccaacggaaaaaaacggaattatcgaatggaatcgaagagaatcatcgaatggacccgaatggaatcatctaatggaatggaatggaataatccatggactcgaatgcaatcatcatcgaatggaatcgaatggaatcatcgaatggactcgaatggaataatcattgaacggcatcgaatggaatcatcatcggatggaaatgaatggaatcatcatcgaatggaatcgaatagaattatggaatgaaatccagtgtgatc

SEQ ID NO: 63 - DNA, 3354 bp, Homo sapiens
ccaacgccaggcagcaaggactgcagcgtgcctacctgtgcagctgcaacccagcgtgcgggagggctgtcgcctcgcccccacttgctcttaatgacccagtgatgggaaaagggacccagccctcaaaggcagggctgacagctgagcgctctcaaccacgcacccaaattagaagctgctgggtcggcagaaaggctaaagggaggcgcccgagggctgaggttaccgtcctccagaacaggtctggccacggcggagcgcgccacggcgtgcccgggcaggctagtgccagcctgcaggccccgcggcgctggtgcctccgacaagtatttgctgagcgcctactgcgtactaggcgccgccgaggggagggcagacccgggcagcgccccgcacccccggcggggaaccgggggcatctttcagccacagaaagctggagaagacagaggagctcctgggaagcagggactgagcgacaggaaggggccgagaagcggcgcgggagacccggagagggaaaaggcactggggctgaggcccccggcctggtccgcgacctgtgatgctgaatcgggggtgcccgggcgtgccgtggccgcggccgcctcctcccagacgcccccgggtgtgagggcgccgggcccgaggctcccgggtacgccggcgtggggaccgtgcccagcgcgaggccacgggtggggcccggattcccgcaggccccagggaggaaggggcccccgcccgccgcagcccccgacgcccgctcacctgtgcccgcgggccccgcccggccccacccacccgccgccgccgccgccgccgccgcttacgcccgccggccccgcgcccccggcccgcgccgcgcgtattgctgccgcctgggggcgaggagggcgcgcggcccggccgatccctgcccgcactcaccgttcacaggcgcgactgcccccggggccagggccggggccgaggccggggcggggcgggggcgggggcgcgcggttcgccccgcgcatgggctccgtccgcggcgggtgcggctcgggttgcgggcgcagggcacgggcggcggagactcgggcgggcctgcgcacgccccgccccgcgcccgtccgtctgccaggcgcggcctaccattggctgcgcgccatcgggccccgccccaccccggttggctgagcggcccgtctgtcaggagccgcggtcgggcggggcttccgggagcaacgcgggaggcggagccagtagggccgcggcctctcggggttgggcttggctggagaccggagccgagctcggggttgctcgaggaaggccagggagccggtgtctgggggcccggggcggcatctccgagcagggccccgggctctcccgggaacaggccggcgagagaacccgactcagcggtgccggtgcaccagaggccctccctgcgccggcagcgcggcgccgcccaccgcggaggtcccggggctacgggctggggaaaggctgggatccgccgggaccaaggcgggatgctcggagctgggggcccccgggtggccgcggggtccggttgcccggctgcccttccgcgcaggtggagcggccgcgcaccccacctaccaccacgcaccccagctcccctactcccaccgcaacccaccccgaggacgcttgcagccccggtggggttccgggcggcgggcgcagccgtgtgccctggggccaggcgtgagaacgccccctccaccactctcctctttctcgggcctgcgtggcaggcgaccctgcccgcccagtccccccaaacttgaggttccaacgctgcagaagctcagcgtggtccagttaaaccgtacccacaagttgccacaggggagcgaagtgcccagggctcaccccaagcacatagacgcacatcctagctctttcaaacccaaaaagacatgtttttaacattttttaaaattgcaaaggaatcagaaatacatcctcattaaaaaacaaaaagggccgggcgcggtggctcacgcctataatcccagcacttcgggaggcctaagcgggtggattacttgaggtcaggagttcaagaccagcctggccaacatgatgaaaccctgtctctactaaaaacacaaaaaattagtcggctgcagtggcgcgcgcctgtagtcccagctactcgggaggctgaggcaggagaatcgctggaacccgggaggcggatgttgcggtgagccgaaatcgcgccactgcactgggcaacagagcgctacttcgtctcaaaacaaaaaaggcgttttacagtcaggcaaaccctgcctctgcccagtccagcgcccgcctcgccctcctgcgccggtcgctgctgacccggggctccacccacgtgcggtcccgggggtcccgcctgccgtccgtccaccgcgcggtcgcagtcagagctcggctcggggcggacacgcatgaacgcgagtgagaagcggcgactggacggcgggcggcagcgtgtcccgcgggccgggcactcgggtgcgctccggcgtccggtgtgtgtttcctggtcctcgggggcgcttcccccggtgcttccttttgccgtcggcctcacttccaaccgaaggtcaggacggcaggcctcggccccaggggcgacccttccacctgggaaaggtgggcgcgagcctccagcagagaccgcctttacccgccccgcgtgggaagcgccaggatcgcgaggaaacgcgacacgtgcatcgcgacggcccaggcacggagccgcaggaagctggcacctgacgcgcctgcgcccacccaactcgagttggtggcgcgtcaccttcccctgggatcgcccgcagcaggggcgcccacgcacgtgccagtccacgtggccccgccctagcgaccgttgctaaggggcgtggctcagccgcacggaacccgagcccccggcgacttataaatatttgcgtattcaaatgaggcctggctcccgttgctatggcgcccaggccgcaaccccgcggcggccggaagaacagcctggagtaggagacagcgcctggaggtggagggcgcccagggccgagctgccagggccggacacctaggctgagccctcaggtgagagccgagcgcacccttggggtgggagccgcaagcctcgccctatgaccggtgccaggagggaacctgcgccgaggcgtgggcgcggggacgaagcagcacagccatcggggacccagtgatggccccgcatgtcagatctggtcccctgaggacccttgcctccaccaccccctggccctgcactgaaagggctccctgtcaggagacaggaggggccccaagccc

(19) United States
(12) Patent Application Publication
(10) Pub. No.: US 2009/0246771 A1
(43) Pub. Date: Oct. 1, 2009

(54) COMPOSITIONS AND METHODS COMPRISING BIOMARKERS OF SPERM QUALITY, SEMEN QUALITY AND FERTILITY

(75) Inventors: Peter W. Laird, South Pasadena, CA (US); Sahar Houshdaran, Alhambra, CA (US); Victoria Cortessis, Los Angeles, CA (US); Kimberly D. Siegmund, San Marino, CA (US); Rebecca Z. Sokol, Ventura, CA (US)

Correspondence Address: DAVIS WRIGHT TREMAINE, LLP/Seattle, 1201 Third Avenue, Suite 2200, SEATTLE, WA, 98101-3045, US

(73) Assignee: University of Southern California, Los Angeles, CA (US)

(21) Appl. No.: 12/264,048

(22) Filed: Nov. 3, 2008

Related U.S. Application Data
(60) Provisional application No. 60/985,170, filed on Nov. 2, 2007.

(51) Int. Cl. C12Q 1/68

(52) U.S. Cl. 435/6

(57) ABSTRACT

Provided are compositions and methods for determining or diagnosing abnormal sperm or fertility, comprising: obtaining sperm DNA from a test subject; determining the methylation status of at least one CpG dinucleotide sequence of at least one gene sequence selected from HRAS, NTF3, MT1A, PAX8, DIRAS3, PLAGL1, SFN, SAT2CHRM1, MEST, RNR1, CYP27B1 and ICAM1; and thereby determining or diagnosing abnormal sperm or fertility. Provided are compositions and methods for identifying agents that cause spermatogenic deficits or abnormal sperm fertility, comprising: obtaining human ES-cell derived primordial germ cells; contacting the germ cells or descendants thereof, with a test agent; culturing the contacted cells; determining, using a genomic DNA of the sample, the methylation status of at least one CpG dinucleotide sequence of at least one gene sequence selected from the above group; and identifying at least one test agent that causes at least one of spermatogenic deficits, abnormal sperm, and abnormal fertility.

### CROSS-REFERENCE TO RELATED APPLICATIONS

This application is claims the benefit of priority to U.S. Provisional Patent Application Ser. No. 60/985,170 filed 2 Nov. 2007, and incorporated by reference herein in its entirety.

### FEDERAL FUNDING ACKNOWLEDGEMENT

This work was at least in part supported by the Southern California Environmental Health Sciences Center (grant # 5P30ES007048) funded by the National Institute of Environmental Health Sciences. The United States government therefore has certain rights in the invention.

### FIELD OF THE INVENTION

Particular aspects relate generally to DNA methylation and epigenetic reprogramming during development and gametogenesis, and more particularly to novel and effective epigenetic biomarkers and methods for determining and/or diagnosis of sperm quality, semen quality and fertility, comprising determining the methylation status of at least one CpG dinucleotide sequence of at least one gene sequence selected from HRAS, NTF3, MT1A, PAX8, DIRAS3, PLAGL1, SFN, SAT2CHRM1, MEST, RNR1, CYP27B1 and ICAM1. Additional aspects relate to compositions and methods for identifying and/or screening for agents that cause spermatogenic deficits or abnormal sperm fertility, comprising contacting human (or murine, rat, Etc.) ES-cell derived primordial germ cells with a test agent and determining the methylation status of at least one CpG dinucleotide sequence from at least one sequence as disclosed herein.

### BACKGROUND

Ten to twenty percent of couples attempting pregnancy are infertile. Male-factor infertility accounts entirely for approximately 20% of these cases, and is contributory in an additional 30% [1,2]. Well defined causes of male-factor infertility are known to include congenital and acquired dysfunction of the hypothalamic-pituitary-testicular endocrine axis, anatomic defects, chromosomal abnormalities, and point mutations [3-5]. However, these diagnoses account for only a small proportion of cases, and etiology remains unknown for most male-factor infertility patients [1,2].

The mammalian germ line undergoes extensive epigenetic reprogramming during development and gametogenesis. In males, dramatic chromatin remodeling occurs during spermatogenesis [6,7], and widespread erasure of DNA methylation followed by de novo DNA methylation occurs developmentally in two broad waves [6,8-11]. The first occurs before emergence of the germ line, establishing a pattern of somatic-like DNA hypermethylation in cells of the pre-implantation embryo that are destined to give rise to all cells of the body, including germ cells. The second widespread occurrence of erasure takes place uniquely in primordial germ cells. Subsequent de novo methylation occurs during germ cell maturation and spermatogenesis, establishing a male germ line pattern of DNA methylation that remains hypomethylated compared with somatic cell DNA [8,12-16].

A small number of studies have addressed the epigenetic state of the human male germ line. Substantial variation in DNA methylation profiles is reported in ejaculated sperm of young, apparently healthy men. Notable distinctions were observed both between samples from separate men and among individually assayed sperm from the same man [17].

Although this variation suggests that DNA methylation may be used as a biomarker of sperm quality, semen quality and fertility were not assessed in this study [17].

### SUMMARY OF EXEMPLARY ASPECTS

Male-factor infertility is a common condition, and etiology is unknown for a high proportion of cases. Abnormal epigenetic programming of the germline is disclosed as a mechanism compromising spermatogenesis of some men currently diagnosed with idiopathic infertility. During germ cell maturation and gametogenesis, cells of the germ line undergo extensive epigenetic reprogramming. This process involves widespread erasure of somatic-like patterns of DNA methylation followed by establishment of sex-specific patterns by de novo DNA methylation.

According to particular aspects, incomplete reprogramming of the male germ line results in both altered sperm DNA methylation and compromised spermatogenesis.

Particular aspects provide the first discovery and disclosure ever of a broad epigenetic defect associated with abnormal semen parameters. Additional aspects relate to an underlying mechanism for these broad epigenetic changes, comprising improper erasure of DNA methylation during epigenetic reprogramming of the male germ line.

Concentration, motility and morphology of sperm was determined in semen samples collected by male members of couples attending an infertility clinic. METHYLIGHT™ and ILLUMINA™ assays were used to measure methylation of DNA isolated from purified sperm from the same samples. Methylation at numerous sequences was elevated in DNA from poor quality sperm, and provide novel and effective epigenetic biomarkers of sperm quality, semen quality and fertility.

Particular exemplary aspects, provide methods for determining or diagnosing abnormal sperm or fertility, comprising: obtaining a sample of human sperm DNA from a test subject; determining, using the genomic DNA of the sample, the methylation status of at least one CpG dinucleotide sequence of at least one gene sequence selected from the group consisting of HRAS, NTF3, MT1A, PAX8, DIRAS3, PLAGL1, SFN, SAT2CHRM1, MEST, RNR1, CYP27B1 and ICAM1; and determining, based on the methylation status of the at least one CpG sequence, the presence or diagnosis of abnormal sperm or fertility with respect to the test subject. In certain aspects, the determined methylation status of the at least one CpG sequence is hypermethylation. In particular embodiments, determining the methylation status of at least one CpG dinucleotide sequence comprises treating the genomic DNA, or a fragment thereof, with one or more reagents to convert 5-position unmethylated cytosine bases to uracil or to another base that is detectably dissimilar to cytosine in terms of hybridization properties. Preferably, treating comprises use of bisulfite treatment of the DNA.

In certain aspects, the at least one gene sequence is selected from the group consisting of HRAS SEQ ID NOS:63 and 20, NTF3 SEQ ID NOS:2 and 14, MT1A SEQ ID NOS:4 and 16, PAX8 SEQ ID NOS:1 and 13, DIRAS3 SEQ ID NOS:3 and 15, PLAGL1 SEQ ID NOS:7 and 19, SFN SEQ ID NOS:6 and 18, SAT2CHRM1 SEQ ID NOS:9 and 21, MEST SEQ ID NOS:5 and 17, RNR1 SEQ ID NOS:10 and 22, CYP27B1 SEQ ID NOS:11 and 23 and ICAM1 SEQ ID NOS:12 and 24.

In particular aspects, abnormal sperm comprises at least one of abnormal sperm concentration, abnormal motility, abnormal total normal morphology, abnormal volume, and abnormal viscosity. In certain embodiments, abnormal sperm comprises at least one of abnormal sperm concentration, abnormal motility, and abnormal total normal morphology.

Certain aspects of the methods, comprise determining, using the genomic DNA of the sample, the methylation status of at least one CpG dinucleotide sequence of at least one gene sequence selected from the group consisting of HRAS, NTF3, MT1A, PAX8 and PLAGL1. In certain embodiments, the at least one gene sequence is selected from the group consisting of HRAS SEQ ID NOS:63 and 20, NTF3 SEQ ID NOS:2 and 14, MT1A SEQ ID NOS:4 and 16, PAX8 SEQ ID NOS:1 and 13, and PLAGL1 SEQ ID NOS:7 and 19.

Yet additional aspects, provide methods for determining or diagnosing abnormal sperm or fertility, comprising: obtaining a sample of human sperm DNA from a test subject; determining, using the genomic DNA of the sample, the methylation status of at least one CpG dinucleotide sequence of at least one gene sequence from each of a repetitive DNA element sequence group, a maternally imprinted gene sequence group, and a non-imprinted gene sequence group; and determining, based on the methylation status of the at least one CpG sequence from each of the groups, the presence or diagnosis of abnormal sperm or fertility with respect to the test subject. In certain implementations, the at least one gene sequence from a repetitive element group comprises at least one selected from the group consisting of SAT2CHRM1 SEQ ID NOS:9 and 21. In certain aspects, the at least one gene sequence from a maternally imprinted gene group comprises at least one selected from the group consisting of PLAGL1 SEQ ID NOS:7 and 19, MEST SEQ ID NOS:5 and 17, and DIRAS3 SEQ ID NOS:3 and 15. In particular embodiments, the at least one gene sequence from a non-imprinted gene group comprises at least one selected from the group consisting of HRAS SEQ ID NOS:63 and 20, NTF3 SEQ ID NOS:2 and 14, MT1A SEQ ID NOS:4 and 16, PAX8 SEQ ID NOS:1 and 13, SFN SEQ ID NOS:6 and 18, RNR1 SEQ ID NOS:10 and 22, CYP27B1 SEQ ID NOS:11 and 23 and ICAM1 SEQ ID NOS:12 and 24.

Yet further aspects provide methods for screening for agents that cause spermatogenic deficits, abnormal sperm or abnormal fertility comprising: obtaining human ES-cell derived primordial germ cells; contacting the germ cells or descendants thereof, with at least one test agent; culturing the contacted germ cells or the descendants thereof under conditions suitable for germ cell proliferation or development; obtaining a sample of genomic DNA from the contacted cultured germ cells or the descendants thereof; determining, using the genomic DNA of the sample, the methylation status of at least one CpG dinucleotide sequence of at least one gene sequence selected from the group consisting of HRAS, NTF3, MT1A, PAX8, DIRAS3, PLAGL1, SFN, SAT2CHRM1, MEST, RNR1, CYP27B1 and ICAM1; and identifying, based on the methylation status of the at least one CpG sequence, at least one test agent that causes at least one of spermatogenic deficits, abnormal sperm, and abnormal fertility. In certain aspects, the determined methylation status of the at least one CpG sequence is hypermethylation. In certain embodiments, the at least one gene sequence is selected from the group consisting of HRAS SEQ ID NOS:63 and 20, NTF3 SEQ ID NOS:2 and 14, MT1A SEQ ID NOS:4 and 16, PAX8 SEQ ID NOS:1 and 13, DIRAS3 SEQ ID NOS:3 and 15, PLAGL1 SEQ ID NOS:7 and 19, SFN SEQ ID NOS:6 and 18, SAT2CHRM1 SEQ ID NOS:9 and 21, MEST SEQ ID NOS:5 and 17, RNR1 SEQ ID NOS:10 and 22, CYP27B1 SEQ ID NOS:11 and 23 and ICAM1 SEQ ID NOS:12 and 24.

### BRIEF DESCRIPTION OF THE DRAWINGS

FIG. 1 shows, according to particular exemplary aspects, box plots illustrating associations between semen parameters and level of methylation (PMR) in DNA isolated from 65 study sperm samples. DNA methylation was measured by MethyLight. Methylation targets were sequences specific to the genes HRAS, NTF3, MT1A, PAX8, PLAGL1, DIRAS3, MEST and SFN and the repetitive element Satellite 2 (SAT2CHRM1). P-value for trend over category of semen parameter is given for each plot. Rows: DNA methylation targets; columns: semen parameters.

FIG. 2 shows, according to particular exemplary aspects, cluster analysis of 36 MethyLight targets in 65 study sperm DNA samples. Left: dendrogram defining clusters; rows: 35 methylation targets; columns: 65 study samples ordered left to right on sperm concentration (samples A-G were also included in Illumina analyses (see FIG. 3)) with poor to good concentration (blue), motility (purple), and morphology (green) represented by darkest to lightest hue; body of figure: standardized PMR values represented lowest to highest as yellow to red. X=missing.

FIG. 3 shows, according to particular exemplary aspects, Results of Illumina analysis of 1,421 autosomal sequences in DNA isolated from sperm and buffy coat. Seven study sperm samples (A-G; ordered left to right on sperm concentration), screening sperm (S), two buffy coat (1-2). Level of DNA methylation scored as β-value. Color: β-value for column sample at row sequence (green: βP<0.1; yellow: 0.1≦β≦0.25; orange 0.25<β≦0.5; red: β>0.5). Ml and PI: maternally and paternally imprinted genes (black bar). Sequences assigned to tertile of median β-value among buffy coat DNA samples (I, II, III) and sorted within tertile on median βP-value among sperm DNA samples. Box 1: sequences with sperm-specific DNA methylation; Box 2: sequences with buffy coat-specific DNA methylation.

### DETAILED DESCRIPTION OF EXEMPLARY EMBODIMENTS

Overview. There have been several prior art attempts in the art to assess sperm DNA methylation together with either sperm quality or fertility outcomes. However, the measures of DNA methylation used were limited, consisting of either a nonspecific genome-wide measure [18], or small and specialized subsets of DNA methylation targets [19-21].

Specifically, in the only study prior art study addressing the relationship between DNA methylation and fertility outcomes, immunostaining was used to measure genome-wide levels of DNA methylation in samples of ejaculated sperm collected for conventional in vitro fertilization (IVF) [18], and no association was observed between sperm DNA methylation and either fertilization rate or embryo quality in 63 IVF cycles. There was, however, a possible association with pregnancy rate after transfer of good quality embryos. Interpretation of these results is limited by both small sample size and the use of a single summary measure of genome-wide DNA methylation.

Moreover, with respect to the prior art studies [19-21] with small and specialized subsets of DNA methylation targets, sequence-specific measures were used to investigate the relationship between methylation of human sperm DNA and spermatogenesis. One study assessed DNA from spermatogonia and spermatocytes microdissected from seminiferous tubules of biopsied testicular tissue with spermatogenic arrest. DNA profiles consistent with correctly established paternal imprints were reported in all samples [19]. In the remaining two studies [20 and 21], DNA profiles were measured at specific DMRs associated with each of two genes, one paternally and one materially imprinted, and the resulting profiles were related to concentration of ejaculated sperm, an indicator of sperm quality. One of these studies reported correctly erased maternal imprints and correctly established paternal imprints in DNA from sperm of low concentration [21]. By contrast, the second reported that although maternal imprinting of MEST was correctly erased in DNA from sperm of low concentration, methylation at an H19 sequence typically de novo methylated in spermatogenesis was incomplete in these samples [20]. No compelling explanation was offered for the apparently differing results of these studies. It is noteworthy, however, that each addressed sequences of only one or two imprinted genes, an extremely small and specialized subset of DNA methylation targets in the human genome. Data from these published studies could not, therefore, have revealed a disruption involving large numbers of genes, or shown that genes that are not imprinted are also affected.

Particular aspects provide methods for determining or diagnosing abnormal sperm or fertility, comprising: obtaining a sample of human sperm DNA from a test subject; determining, using the genomic DNA of the sample, the methylation status of at least one CpG dinucleotide sequence of at least one gene sequence selected from the group consisting of HRAS, NTF3, MT1A, PAX8, DIRAS3, PLAGL1, SFN, SAT2CHRM1, MEST, RNR1, CYP27B1 and ICAM1; and determining, based on the methylation status of the at least one CpG sequence, the presence or diagnosis of abnormal sperm or fertility with respect to the test subject. In certain embodiments the at least one gene sequence is selected from the group consisting of HRAS SEQ ID NOS:63 and 20, NTF3 SEQ ID NOS:2 and 14, MT1A SEQ ID NOS:4 and 16, PAX8 SEQ ID NOS:1 and 13, DIRAS3 SEQ ID NOS:3 and 15, PLAGL1 SEQ ID NOS:7 and 19, SFN SEQ ID NOS:6 and 18, SAT2CHRM1 SEQ ID NOS:9 and 21, MEST SEQ ID NOS:5 and 17, RNR1 SEQ ID NOS:10 and 22, CYP27B1 SEQ ID NOS:11 and 23 and ICAM1 SEQ ID NOS:12 and 24.

In particular aspects at least on CpG dinucleotide sequence within an amplicon is determined. In preferred aspects, the at least one amplicon sequence is selected from the group consisting of: HRAS SEQ ID NOS:20, NTF3 SEQ ID NO: 14, MT1A SEQ ID NO:16, PAX8 SEQ ID NO:13, DIRAS3 SEQ ID NO:15, PLAGL1 SEQ ID NO:19, SFN SEQ ID NO:18, SAT2CHRM1 SEQ ID NO:21, MEST SEQ ID NO:17, RNR1 SEQ ID NO:22, CYP27B1 SEQ ID NO:23 and ICAM1 SEQ ID NO:24.

Preferably, the amplicon is part of a contiguous CpG island sequence. In preferred aspects, the CpG island sequence is selected from the group consisting of: HRAS SEQ ID NOS:63, NTF3 SEQ ID NO:2, MT1A SEQ ID NO:4, PAX8 SEQ ID NO:1, DIRAS3 SEQ ID NO:3, PLAGL1 SEQ ID NO:7, SFN SEQ ID NO:6, SAT2CHRM1 SEQ ID NO:9, MEST SEQ ID NO:5, RNR1 SEQ ID NO:10, CYP27B1 SEQ ID NO:11 and ICAM1 SEQ ID NO:12.

Coordinate methylation within CpG islands. According to particular aspects, and as recognized in the relevant art, hypermethylation is coordinate within a CpG island. For Example, data (see Eckhardt et al., Nat Genet. 2006 December; 38(12):1378-85. Epub 2006 Oct. 29; incorporated by reference herein in its entirety) has been generated by analyzing methylation (using bisulfite sequencing) in CG-rich regions across entire chromosomes to provide a methylation map of the human genome (at least of the CPG rich regions thereof). To date, these data comprise methylation data of 3 complete human chromosomes (22, 20, and 6) for a variety of different tissues and cell types. Based on these data, for methylation patterns within CpG dense regions, methylation is typically found to be either present for all methylatable cytosines or none. This methylation characteristic or pattern is referred to in the art as “co-methylation” or “coordinate methylation.” The findings of this paper support a “significant correlation” of comethylation over the distance of at least 1,000 nucleotides in each direction from a particular determined CpG within a CpG dense region (see, e.g., page 2, column 2, 1^{st} full paragraph, of Eckhardt et al publication document). Furthermore, such co-methylation forms the basis for long-standing common methods such as MSP and particular MethyLight embodiments that rely on such co-methylation (e.g., as employed herein, the primers and/or probes each typically encompass multiple CpG sequences), and has now been further confirmed over entire chromosomes by Eckhardt et al. Therefore, in view of the teachings of the present specification, there is a reasonable correlation between the claimed coordinately methylated sequences, and the recited methods and exemplary methylation marker sequences.

### Measurement of DNA Methylation of the Genomic DNA of Spermatozoa at CpG Islands, DMRs of Imprinted Genes and Repetitive Elements

The present specification describes and discloses the first study ever to investigate the epigenetic state of abnormal human sperm using an extensive panel of DNA methylation assays. Abnormal epigenetic programming of the germ line is herein disclosed as a mechanism compromising fertility of particular men currently diagnosed with idiopathic infertility. Aspects of the present invention indicate that one or more epigenetic processes lead to abnormal spermatogenesis and compromised sperm function.

To assess sperm DNA, methylation at specific targets that are both more numerous and less specialized, a relatively large set of sequence-specific assays was selected for use in the presently disclosed studies and invention.

Specifically, DNA methylation was measured in ejaculated spermatozoa-interrogating sequences in repetitive elements, promoter CpG islands, and differentially methylated regions (DMRs) of imprinted genes. Then, to address the possible role of epigenetic programming in abnormal human spermatogenesis, sequence-specific levels of DNA methylation were related to standard measures of sperm quality.

Applicants' observations indicate a broad epigenetic abnormality of poor quality human sperm in which levels of DNA methylation are elevated at numerous sequences in several genomic contexts. Previous studies of DNA methylation in poor quality sperm interrogated only imprinted loci, measuring methylation of sequences in only one or two genes [19-21].

Aspects of the present invention provide, inter alia, compositions and methods having substantial utility for diagnosing or determining the presence of abnormal sperm or fertility (e.g., comprising at least one of abnormal sperm concentration, abnormal total normal morphology, abnormal motility, abnormal volume, and abnormal viscosity).

As described in the working Example 1, herein below, Applicants initially evaluated 294 MethyLight reactions for the presence of methylation in sperm DNA from an anonymous semen sample obtained from a sperm bank. Standard semen analysis was then conducted on samples collected by 69 men during clinical evaluation of couples with infertility. Thirty seven selected MethyLight reactions were used to assay sperm DNA from 65 of the study samples.

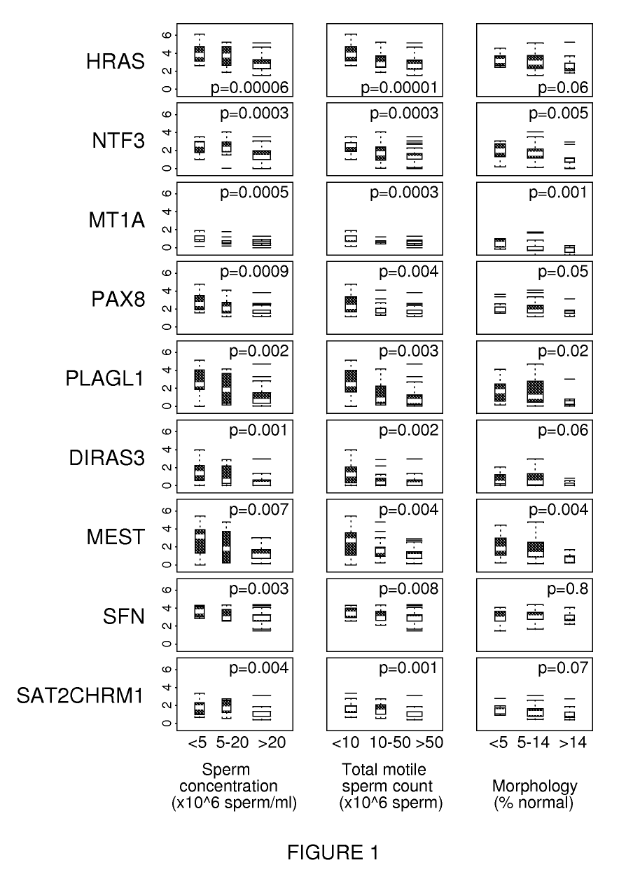

At many of the 37 sequences, methylation levels were elevated in DNA from poor quality sperm. For example, striking associations with each of sperm concentration, motility and morphology were observed for five sequences: HRAS, NTF3, MT1A, PAX8 and the maternally imprinted gene PLAGL1 (FIG. 1). Applicants also found elevated DNA methylation to be significantly associated with poor semen parameters for the DIRAS3 and MEST maternally imprinted genes (FIG. 1).

Associations between results of each of the 37 MethyLight assays and sperm concentration were highly significant for HRAS, NTF3, MT1A, PAX8, DIRAS3 and PLAGL1 and were also significant (somewhat less) for SFN, SAT2CHRM1 and MEST (see Table 1 of Example 1, and see also FIG. 1).

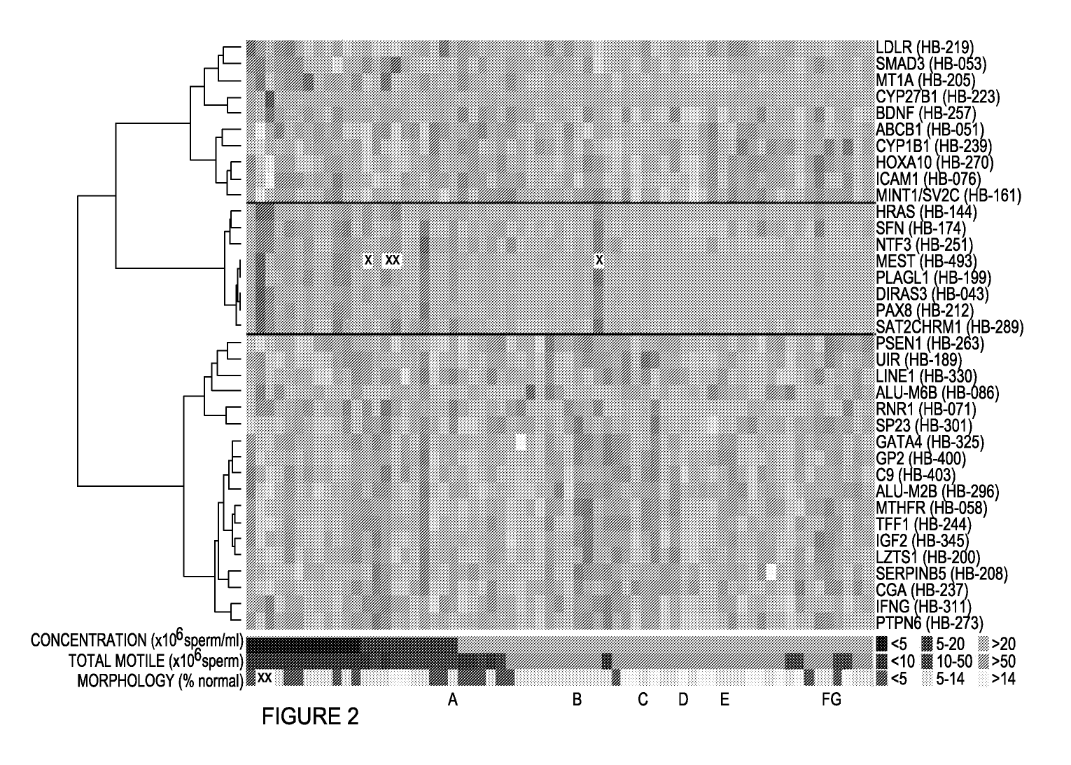

Unsupervised cluster analysis identified three distinct clusters of sequences based on DNA methylation profiles in the 65 samples (FIG. 2). The middle cluster shown in FIG. 2 includes eight of the above nine sequences (all except MT1A) individually associated with semen parameters, and includes not only three sequences that are differentially methylated on imprinted loci, but also three single copy sequences specific to non-imprinted genes, and a repetitive element, Satellite 2 (referred to herein as SAT2CHRM1).

Significantly, this surprising result indicates that sperm abnormalities may be associated with a broad epigenetic defect of elevated DNA methylation at numerous sequences of diverse types, rather than a defect of imprinting alone as previously suggested [20].

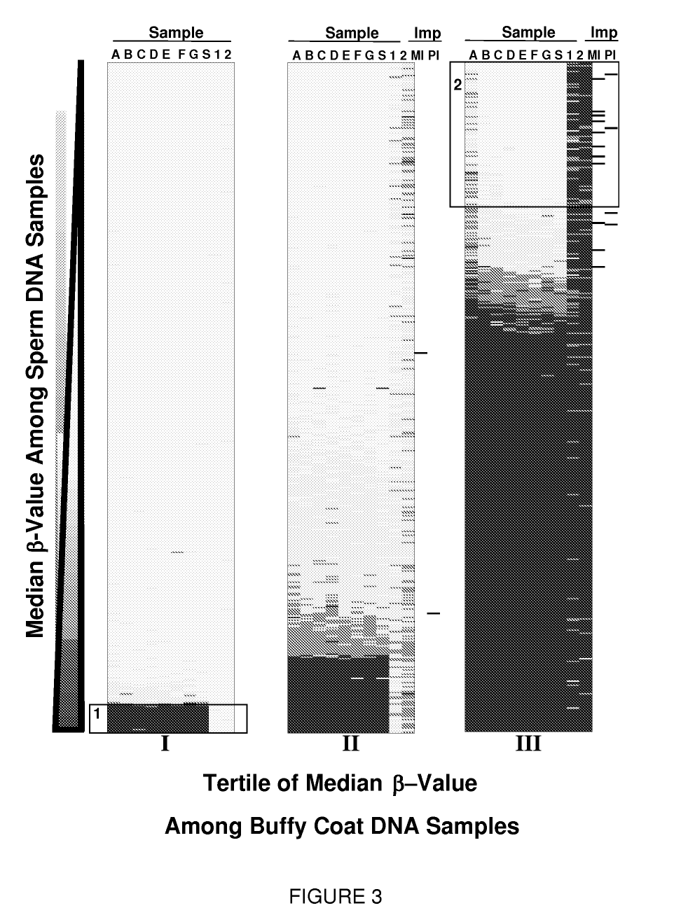

To learn more about the possible extent of this apparent defect, the ILLUMINA™ platform was used to conduct DNA methylation analysis of 1,421 sequences in autosomal loci (discussed in more detail under Example 1 herein below). Briefly, the results of the ILLUMINA™ analyses appear in FIG. 3. Box 1 of FIG. 3 identifies 19 sequences with sperm-specific DNA methylation.

Various semen parameters have been correlated herein with abnormal DNA methylation (sperm concentration; total normal morphology; motility, volume, viscosity, etc.). According to preferred aspects, three of these semen parameters show the highest correlations with abnormal DNA methylation: sperm concentration; total normal morphology; and motility. FIG. 2, for example, shows that the corresponding MLL reactions are clustered based on sperm concentration.

Particular aspects of the present invention, therefore, provide marker(s) and marker subsets having utility for determining at least one of (A) abnormal sperm concentration, (B) abnormal morphology, and (C) abnormal motility. With respect to (A), abnormal sperm concentration, markers are provided in the following order of statistical significance from left to right, based on the p-value: HRAS, NTF3, MT1A, PAX8, DIRAS3, PLAGL1, SFN, SAT2CHRM1, MEST, RNR1, and CYP27B1. Nine of these markers have p-values well below 0.05, and therefore are very significant. Additionally provided are the markers, RNR1 and CYP27B1, both having p-values of 0.02, and therefore also provide for utility in this respect.

With respect to (B), abnormal total motile sperm, markers are provided in the following order of statistical significance from left to right, based on the p-value: HRAS, NTF3, MT1A (NTF3 and MT1A equally significant), SAT2CHRM1, DIRAS3, PLAGL1, MEST, PAX8, and SFN. These markers have p-values well below 0.05, and therefore are very significant. Additionally provided are the markers: RNR1 (p-value 0.04) and CYP27B1 and BDNF (both with p-value of 0.05), and therefore also provide for utility in this respect.

With respect to (C), abnormal motility, markers are provided in the following order of statistical significance from left to right, based on the p-value: MT A, MEST, NTF3, PLAGL1. Additionally provided are the markers PAX8 AND ICAM1 (both having p-values of 0.05), and therefore also provide for utility in this respect.

### Improper Erasure of Pre-Existing Methylation

According to particular aspects, only sequence-specific measures of DNA methylation are expected to reveal variation at individual sites, because of the enormous number of methylation targets in the human genome. These include millions of repetitive DNA elements for which methylation is postulated to silence parasitic and transposable activity. There are also large numbers of target sequences corresponding to single copy genes. Examples include thousands of promoter CpG islands for which methylation appears to mediate expression of genes in a tissue- and lineage-specific fashion, and DMRs associated with dozens of imprinted genes for which parent-of-origin DNA methylation marks are believed to mediate monoallelic expression in somatic cells.

As disclosed herein, Applicants' high-throughput analysis addressed hundreds of DNA methylation targets, and was thus designed to reveal methylation defects.

Elevated DNA methylation could, in theory, arise from either de novo methylation or improper erasure of pre-existing methylation. Although Applicants cannot rule out the possibility that processes responsible for de novo methylation are inappropriately activated in abnormal spermatogenesis, according to particular aspects, disruption of erasure is most likely the primary mechanism underlying abnormal spermatogenesis. Widespread erasure of DNA methylation occurs in both the pre-implantation embryo and again, uniquely, in primordial germ cells around the time that they enter the genital ridge. Several factors point to disruption of the second erasure as underlying the defect(s) described herein. Primordial germ cells arise from cells of the proximal epiblast which have themselves embarked upon somatic development, as shown by expression of somatic genes [25,26]. The germ cell lineage must therefore suppress the somatic program, which in mice is accomplished in part by genome-wide erasure of DNA methylation soon after germ cells migrate to the genital ridge [27]. This erasure affects DNA methylation on single copy genes, imprinted genes and repetitive elements [27]. Therefore, disruption of the second, genital ridge erasure most likely results in the type of pattern we observe in poor quality sperm, with elevated levels of DNA methylation at DNA sequences of each of these sequence types. Further, because this second erasure is confined to primordial germ cells, Applicants further reasoned that its disruption would be compatible with normal somatic development.

In humans, primordial germ cells colonize the genital ridge at about 4.5 weeks of gestation. Applicants are not aware of data describing DNA methylation in the human germ line at this date; however, the DMR in MEST at which Applicants found elevated DNA methylation in poor quality sperm is reportedly unmethylated in the male germ line by week 24 of gestation [28]. Potential causes of disrupted erasure have not been investigated. However, weeks 4.5-24 of gestation represent post-implantation stages of development wherein fetal physiology may be influenced by maternal factors and environmental compounds that cross the placenta. Possible origins of male infertility as early as 4.5 weeks of human gestation have not been studied. However, transient in vivo chemical exposure at 7-15 days post conception, which includes the analogous stage of murine development [29,30], results in spermatogenic deficits in rats with grossly normal testes [31] and may be associated with elevated methylation of sperm DNA [32].

Taken together, the observations disclosed herein indicate for the first time that epigenetic mechanisms contribute to a substantial portion of male factor infertility, and provide novel compositions and methods for the diagnosis, detection or determination of abnormal sperm or fertility. Also provided are methods for screening for agents that cause spermatogenic deficits, abnormal sperm or fertility comprising: obtaining human ES-cell derived primordial germ cells; contacting the germ cells with at least one test agent; culturing the contacted germ cells; obtaining a sample of genomic DNA from the contacted cultured germ cells; determining, using the genomic DNA of the sample, the methylation status of at least one CpG dinucleotide sequence of at least one gene sequence selected from the group consisting of HRAS, NTF3, MT1A, PAX8, DIRAS3, PLAGL1, SFN, SAT2CHRM1, MEST, RNR1, CYP27B1 and ICAM1; and identifying, based on the methylation status of the at least one CpG sequence, at least one test agent that causes spermatogenic deficits, abnormal sperm or fertility.

### Example 1

### Sequence-Specific Levels of DNA Methylation were Related to Standard Measures of Sperm Quality

Overview. This is the first study ever to describe the epigenetic state of abnormal human sperm using an extensive panel of DNA methylation assays. To assess sperm DNA methylation at specific targets that are both more numerous and less specialized, a relatively larger set of sequence-specific assays was selected for use in the present study. DNA methylation was measured in ejaculated spermatozoa-interrogating sequences in repetitive elements, promoter CpG islands, and differentially methylated regions (DMRs) of imprinted genes. Then, to address the possible role of epigenetic programming in abnormal human spermatogenesis, sequence-specific levels of DNA methylation were related to standard measures of sperm quality.

### Materials and Methods

Semen samples. Study semen samples were collected by 69 consecutive men ages 22-49 years who were partners of women undergoing evaluation for infertility at the Endocrine/Infertility Clinic of the Los Angeles County/University of Southern California Keck School of Medicine Medical Center. One additional semen sample was obtained from a sperm bank. The study was approved by the Institutional Review Board of the University of Southern California. Informed consent was not required because this research involved stored materials that had previously been collected solely for non-research purposes and were anonymous to the researchers/authors.

Semen Analysis. Standard semen analysis was performed using WHO criteria and Strict Morphology as previously described [33,34]. Semen volume, sperm concentration and motility, and leukocyte count were measured using the MicroCell chamber (Conception Technologies, San Diego, Calif.). Sperm morphology was assessed with the use of prestained slides (TestSimplets, Spectrum Technologies, Healdsburgh, Calif.), and percentage of morphologically normal sperm was documented. The samples were categorized according to concentration (<5, 5-20, >20 million sperm/ml), motility (<10, 10-50, >50 total motile sperm count (×10⁶)), and morphology (<5%, 5-14%, >14% normal) of sperm [33,35]. Presence of any white blood cells, round cells, or epithelial cells was recorded. Following semen analysis, samples were stored at −30° C. until processing for molecular analysis.

Sperm Separation from Seminal Plasma. Semen samples were allowed to thaw at 37° C. Sperm were separated from seminal plasma using ISOLATE® Sperm Separation Medium (Irvine Scientific, Santa Ana, Calif.), a density gradient centrifugation column designed to separate cellular contaminants (including leukocytes, round cells, and miscellaneous debris) from spermatozoa [24]. Separation was performed according to the manufacturer's protocol [36], and the purity of separated sperm from contaminating cells was documented by light microscopy.

DNA isolation. DNA was isolated from purified sperm as previously described [37], with 0.1×SSC added to the Lysis buffer, and samples incubated at 55° C. over night or longer to complete the lysis procedure.

Laboratory Analysis of DNA Methylation. Sodium bisulfite conversion was performed as previously described [23]. The amount of DNA in each aliquot was normalized, and a bisulfite-dependent, DNA methylation-independent control reaction was performed to confirm relative amounts of DNA in each sample. METHYLIGHT™ analyses were performed as previously described [23]. Reaction IDs and sequences of the primers and probes used in the 294 METHYLIGHT™ reactions are as previously published (see Table S1 (Sections A-B): doi:10.1371/journal.pone.0001289.s001 (0.10 MB PDF; incorporated by reference herein in its entirety). Additionally, according to particular aspects of the present invention, names of preferred markers and respective primers, probes and genomic sequences corresponding to the respective amplicons are listed below in TABLE 1.

TABLE 1
Primers and Probes for exemplary preferred MethyLight Assays.

| | | | | Genomic sequence |
|---|---|---|---|---|
| | Forward | Reverse | Probe Oligo | corresponding to |
| | Primer | Primer | Sequence | amplicon Sequence |
| Gene | (SEQ ID NO:) | (SEQ ID NO:) | (SEQ ID NO:) | (SEQ ID NO:) |
| HRAS | GAGCGATGACG | CGTCCACAAAA | 6FAM- | CGTCCACAAAATGGTTCTGG |
| | GAATATAAGTT | TAATTCTAAAT | CACTCTTACCC | ATCAGCTGGATGGTCAGCGC |
| | GG | CAACTAA | ACACCGCCGAC | ACTCTTGCCCACACCGCCGG |
| | (SEQ ID | (SEQ ID | G-BHQ-1 | CGCCCACCACCACCAGCTTA |
| | NO: 46) | NO: 47) | (SEQ ID | TATTCCGTCATCGCTC |
| | | | NO: 48) | (SEQ ID NO: 20) |
| NTF3 | TTTCGTTTTTG | CCGTTTCCGCC | 6FAM- | CCCCGCCCTTGTATCTCATG |
| | TATTTTATGGA | GTAATATTC | TCGCCACCACG | GAGGATTACGTGGGCAGCCC |
| | GGATT | (SEQ ID | AAACTACCCAC | CGTGGTGGCGAACAGAACAT |
| | (SEQ ID | NO: 29) | G-BHQ-1 | CACGGCGGAAACGG |
| | NO: 28) | | (SEQ ID | (SEQ ID NO: 14) |
| | | NO: 30) | | |
| MT1A | CGTGTTTTCGT | CTCGCTATCGC | 6FAM- | CGTGTTCCCGTGTTACTGTG |
| | GTTATTGTGTA | CTTACCTATCC | TCCACACCTAA | TACGGAGTAGTGGGTCCGAG |
| | CG | (SEQ ID | ATCCCTCGAAC | GGACCTAGGTGTGGACAGGG |
| | (SEQ ID | NO: 35) | CCACT-BHQ-1 | ACAGGCAAGGCGACAGCGAG |
| | NO: 34) | | (SEQ ID | (SEQ ID NO: 16) |
| | | | NO: 36) | |
| PAX8 | CGGGATTTTTT | ACCTTTCCCCA | 6 FAM- | CGGGACCTCCCTGTCGTACC |
| | TGTCGTATTTG | TACTACCTCCG | ACGAACAATTC | TGAGAGGAGGGCCTGGCCCG |
| | A | (SEQ ID | ACGAACCAAAC | TGAACTGCCCGTACACGGAG |
| | (SEQ ID | NO: 26) | CCTCCT-BHQ-1 | GCAGCATGGGGAAAGGC |
| | NO: 25) | | (SEQ ID | (SEQ ID NO: 13) |
| | | | NO: 27) | |
| DIRAS3 | GCGTAAGCGGA | CCGCGATTTTA | 6 FAM- | GCGCAAGCGGAATCTATGCC |
| | ATTTATGTTTGT | TATTCCGACTT | CGCACAAAAAC | TGTTACCCACACTCCCTGCG |
| | (SEQ ID | (SEQ ID | GAAATACGAAA | CCCCCGCACCCCGCTCCTGT |
| | NO: 31) | NO: 32) | ACGCAAA- | GCGCAAGTCGGAATATAAAA |
| | | | BHQ-1 | CCGCGG |
| | | | (SEQ ID | (SEQ ID NO: 15) |
| | | | NO: 33) | |
| PLAGL1 | ATCGACGGGTT | CTCGACGCAAC | 6FAM- | ACCGACGGGCTGAATGACAA |
| | GAATGATAAATG | CATCCTCTT | ACTACCGCGAA | ATGGCAGATGCCGTGGGCTT |
| | (SEQ ID | (SEQ ID | CGACAAAACCC | TGCCGCCCGCGGCAGCCAAG |
| | NO: 43) | NO: 44) | ACG-BHQ-1 | AGGATGGCTGCGCCGAG |
| | | | (SEQ ID | (SEQ ID NO: 19) |
| | | | NO: 45) | |
| SFN | GAGGAGGGTTC | ATCGCACACGC | 6FAM- | GAGGAGGGCTCGGAGGAGAA |
| | GGAGGAGAA | CCTAAAACT | TCTCCCGATAC | GGGGCCCGAGGTGCGTGAGT |
| | (SEQ ID | (SEQ ID | TCACGCACCTC | ACCGGGAGAAGGTGGAGACT |
| | NO: 40) | NO: 41) | GAA-BHQ-1 | GAGCTCCAGGGCGTGTGCGA |
| | | | (SEQ ID | C |
| | | | NO: 42) | (SEQ ID NO: 18) |
| SAT2CHR | TCGAATGGAAT | CCATTCGAATC | 6FAM- | TCGAATGGAATCAACATCCA |
| M1 | TAATATTTAAC | CATTCGATAAT | CGATTCCATTC | ACGGAAAAAAACGGAATTAT |
| | GGAAAA | TCT | GATAATTCCGT | CGAATGGAATCGAAGAGAAT |
| | (SEQ ID | (SEQ ID | TT-MGBNFQ | CATCGAATGGACCCGAATGG |
| | NO: 49) | NO: 50) | (SEQ ID | (SEQ ID NO: 21) |
| | | | NO: 51) | |
| MEST | CGGCGTTCGGT | CACACTCACCT | 6 FAM- | CGGCGCCCGGTGCTCTGCAA |
| | GTTTTGTAA | ACGAAAACGAT | ACGCACCATAA | CGCTGCGGCGGGCGGCATGG |
| | (SEQ ID | CTC | CCGCGTTATCC | GATAACGCGGCCATGGTGCG |
| | NO: 37) | (SEQ ID | CATACC-BHQ-1 | CCGAGATCGCCTCCGCAGGT |
| | | NO: 38) | (SEQ ID | GAGTGTG |
| | | | NO: 39) | (SEQ ID NO: 17) |
| RNR1 | CGTTTTGGAGA | AAACAACGCCG | 6 FAM- | CGCTCTGGAGACACGGGCCG |
| | TACGGGTCG | AACCGAA | ACCGCCCGTAC | GCCCCCTGCGTGTGGCACGG |
| | (SEQ ID | (SEQ ID | CACACGCAAA- | GCGGCCGGGAGGGCGTCCCC |
| | NO: 52) | NO: 53) | BHQ-1 | GGCCCGGCGCTGCTC |
| | | | (SEQ ID | (SEQ ID NO:22) |
| | | | NO: 54) | |
| CYP27B1 | GGGATAGTTAG | CCGAATATAAC | 6FAM- | GGGACAGCCAGAGAGAACGG |
| | AGAGAACGGAT | CACACCGCC | CCAACCTCAAC | ATGCCCATGAAATAAGGAAA |
| | GTTT | (SEQ ID | TCGCCTTTTCC | AGGCGAGTTGAGGCTGGGGG |
| | (SEQ ID | NO: 56) | TTATTTCA- | CGGTGTGGCTACACTCGG |
| | NO: 55) | | BHQ-1 | (SEQ ID NO: 23) |
| | | | (SEQ ID | |
| | | | NO: 57) | |
| ICAM1 | GGTTAGCGAGG | TCCCCTCCGAA | 6 FAM- | GGCCAGCGAGGGAGGATGAC |
| | GAGGATGATT | ACAAATACTAC | TTCCGAACTAA | CCTCTCGGCCCGGGCACCCT |
| | (SEQ ID | AA | CAAAATACCCG | GTCAGTCCGGAAATAACTGC |
| | NO: 58) | (SEQ ID | AACCGAAA- | AGCATTTGTTCCGGAGGGGA |
| | | NO: 59) | BHQ-1 | (SEQ ID NO: 24) |
| | | | (SEQ ID | |
| | | | NO: 60) | |

Thirty-five METHYLIGHT™ reactions were selected for analysis of study sperm DNA samples based on cycle threshold (C(t)) values from analysis of the anonymous sample of sperm DNA. In brief, C(t) value is the PCR cycle number at which the emitted fluorescence is detectable above background levels. The C(t) value is inversely proportional to the amount of each methylated locus in the PCR reaction well, such that a low C(t) value suggests that the interrogated sequence is highly methylated. C(t) values of 35 or less were interpreted as an indication that a given sequence was methylated in the anonymous sample and selected 33 reactions on this basis. Three additional reactions were included, for which C(t) values slightly exceeded 35. Two (CYP27B1 and HOXA10) were selected based on gene function potentially related to fertility, and one (a non-CpG island reaction for IFNG) based on prior observation by applicants of hypomethylation in tumor versus normal tissue. When multiple reactions for a single locus resulted in C(t) values of less than 35, we selected only the reaction with the lowest C(t) value. Results of METHYLIGHT™ analysis were scored as PMR values as previously defined [23]. Following METHYLIGHT™ analyses, DNA remained from a subset of abnormal samples with greater sperm concentration. ILLUMINA™ analysis was performed on sodium bisulfite-converted sperm DNA of selected remaining samples, the anonymous semen sample, and purchased buffy coat DNA (HemaCare® Corporation, Van Nuys, Calif.) at the USC Genomics Core. Sodium bisulfite conversion for ILLUMINA™ assay was performed using the EZ-96 DNA Methylation Kit™ (ZYMO Research) according to manufacturer's protocol. Illumina Methods and reagents are as previously described [38]. The primer names and probe IDs are listed as previously published (see Table S2; doi:10.1371/journal.pone.0001289.s002 (0.20 MB PDF; incorporated by reference herein in its entirety), identifying 1,421 autosomal sequences of the GoldenGate Methylation Cancer Panel 1, more fully described elsewhere [39,40]. Results of ILLUMINA™ assays were scored as β-values [38]. Relevant amplicons and CpG islands are provided below in TABLE 2 below.

Statistical association analyses of METHYLIGHT™ data. Associations between the ranked METHYLIGHT™ data and categorized semen values (Table 1) were tested using simple linear regression, with the semen characteristic categories scored as 0: low, 1: mid, 2: high. For selected sequences, boxplots of the methylation values (on the log(PMR+1) scale) are shown in FIG. 1. The top and bottom of the box denote the 75^{th} and 25^{th} percentiles, and the white bar the median. Whiskers are drawn to the observation farthest from the box that lies within 1.5 times the distance from the top to the bottom of the box, with values falling outside the whiskers denoted as lines. Results of this analysis were included in FIG. 1 for sequences associated with sperm concentration using the Benjamini and Hochberg procedure [41] to control the false discovery rate at 5%.

TABLE 2
Exemplary, preferred amplicons and CpG islands

| Reaction | HUGO Gene | Previously | Source of | UniGene | Reaction | Alternate Gene |
|---|---|---|---|---|---|---|
| Number | Nomenclature | Published? | published reaction | Number | ID | Name |
| HB-144 | HRAS | Yes | Widschwendter, M. | Hs.37003 | H-HRAS-M1B | V-Ha-ras Harvey rat |
| | | | et al Cancer Res | | | sarcoma viral |
| | | | 64, 3807-3813 (2004) | | | oncogene homolog |
| | | | | | | (HRAS); HRAS 1 |
| HB-251 | NTF3 | Yes | Weisenberger, D. J. | Hs.99171 | H-NTF3-M1B | Neurotrophin 3 |
| | | | et al Nature Genet | | | |
| | | | 38, 787-793 (2006). | | | |
| HB-205 | MT1A | Yes | Weisenberger, D. J. | Hs.655199 | H-MT1A-M1B | Metallothionein |
| | | | et al Nature Genet | | | 1A/Metallothionein-I |
| | | | 38, 787-793 (2006). | | | |
| HB-212 | PAX8 | No | | Hs.469728 | H-PAX8-M3B | Paired Box Gene 8/ |
| | | | | | | PAX8, Paired |
| | | | | | | Domain Gene 8, |
| | | | | | | PPARG Fusion Gene |
| HB-043 | DIRAS3 | Yes | Fiegl, H. et al | Hs.194695 | H-DIRAS3-M1B | Ras homolog gene |
| | | | Cancer Epidemiol | | | family, member |
| | | | BioMark Prev | | | I/NOEY2; DIRAS |
| | | | 13, 882-888 (2004) | | | family, GTP-binding |
| | | | | | | RAS-like 3 (ARHI) |
| HB-199 | PLAGL1 | Yes | Weisenberger, D. J. | Hs.444975 | H-PLAGL1-M1B | Pleiomorphic |
| | | | et al Nature Genet | | | adenoma gene-like |
| | | | 38, 787-793 (2006). | | | 1/LOT1/Zac1 |
| HB-174 | SFN | Yes | Weisenberger, D. J. | Hs.523718 | H-SFN-M1B | Stratifin/14-3-3 |
| | | | et al Nature Genet | | | protein sigma |
| | | | 38, 787-793 (2006). | | | |
| HB-289 | SAT2CHRM1 | Yes | Weisenberger, D. J. | N/A | H-SAT2CHRM1-M1M | SATELLITE 2 |
| | | | et al Nucleic Acids | | | CHROMOSOME 1 |
| | | | Res 33, 6823-6836 (2005) | | | |
| HB-493 | MEST | No | | Hs.270978 | H-MEST-M2B | PEG1 |
| HB-071 | RNR1 | Yes | Muller, H. M. et al. | N/A | H-RNR1-M1B | Ribosomal RNA |
| | | | Cancer Lett209, | | | |
| | | | 231-236 (2004) | | | |
| HB-076 | ICAM1 | Yes | Ehrlich, M. et al. | Hs.643447 | H-ICAM1B-M1B | Intercellular |
| | | | Oncogene 21, | | | adhesion molecule 1 |
| | | | 6694-6702 (2002) | | | (CD54), human |
| | | | | | | rhinovirus receptor |
| HB-223 | CYP27B1 | Yes | Weisenberger, D. J. | Hs.524528 | H-CYB27B1-M1B | cytochrome P450, |
| | | | et al Nature Genet | | | family 27, subfamily |
| | | | 38, 787-793 (2006). | | | B, polypeptide 1 |

| | | | GenBank | mRNA | | | Transcription |
|---|---|---|---|---|---|---|---|
| Reaction | HUGO Gene | Chromosomal | Accession | accession | Parallel/ | Length of | Start (GenBank |
| Number | Nomenclature | Location | Number | number | Antiparallel | Sequence (bp) | Numbering) |
| HB-144 | HRAS | 11p15.5 | AC137894 | NM_176795 | Antiparallel | 165000 | 157238 |
| HB-251 | NTF3 | 12p13 | AC135585 | NM_002527 | Parallel | 35700 | 7048 |
| HB-205 | MT1A | 16q13 | AC106779 | NM_005946 | Parallel | 158297 | 18787 |
| HB-212 | PAX8 | 2q12 | AC016683 | S77905 | Antiparallel | 179937 | 116171 |
| HB-043 | DIRAS3 | 1p31 | AF202543 | U96750 | Parallel | 7242 | 2053 |
| HB-199 | PLAGL1 | 6q24-q25 | AL109755 | U72621 | Antiparallel | 89669 | 53085 |
| HB-174 | SFN | 1p35.3 | AF029081 | BC023552 | Parallel | 10034 | 8563 |
| HB-289 | SAT2CHRM1 | 1 | X72623 | N/A | Parallel | 1352 | N/A |
| HB-493 | MEST | 7q32.2 | NC_000007 | NM_177524 | Parallel | 20084 | 5893 |
| HB-071 | RNR1 | 13p12 | X01547 | N/A | Parallel | 850 | 482 |
| HB-076 | ICAM1 | 19p13.3- | AC011511 | BC015969 | Parallel | 156503 | 85732 |
| | | p13.2 | | | | | |
| HB-223 | CYP27B1 | 12q14.1 | AY288916 | AB005038 | Parallel | 7587 | 1324 |

| | | | | Amplicon Start | Amplicon End | Mean |
|---|---|---|---|---|---|---|
| | | | | Location Relative | Location Relative | Distance from |
| | | Amplicon | Amplicon | to Transcription | to Transcription | Transcription |
| Reaction | HUGO Gene | Location Start | Location End | Start (bp, | Start (bp, | Start (bp, |
| Number | Nomenclature | (GenBank Numbering) | (GenBank Numbering) | GenBank sequence) | GenBank Sequence) | GenBank sequence) |
| HB-144 | HRAS | 156015 | 155920 | 1223 | 1318 | 1271 |
| HB-251 | NTF3 | 7503 | 7576 | 455 | 528 | 492 |
| HB-205 | MT1A | 18175 | 18254 | −612 | −533 | −573 |
| HB-212 | PAX8 | 72708 | 72632 | 43463 | 43539 | 43501 |
| HB-043 | DIRAS3 | 1953 | 2038 | −100 | −15 | −58 |
| HB-199 | PLAGL1 | 53045 | 52969 | 40 | 116 | 78 |
| HB-174 | SFN | 8848 | 8928 | 285 | 365 | 325 |
| HB-289 | SAT2CHRM1 | 1074 | 1153 | N/A | N/A | N/A |
| HB-493 | MEST | 6057 | 6144 | 164 | 251 | 207 |
| HB-071 | RNR1 | 219 | 293 | −263 | −189 | −226 |
| HB-076 | ICAM1 | 85597 | 85676 | −135 | −56 | −96 |
| HB-223 | CYP27B1 | 1728 | 1805 | 404 | 481 | 443 |

| | | Amplicon | Amplicon | UCSC | UCSC | Location of |
|---|---|---|---|---|---|---|
| Reaction | HUGO Gene | Location Start | Location End | Strand | Assembly | Amplicon in Gene |
| Number | Nomenclature | (UCSC Numbering) | (UCSC Numbering) | (+/−) | Date | (e.g., promoter, exon) |
| HB-144 | HRAS | 524232 | 524327 | + | May 2004 | Exon2 |
| HB-251 | NTF3 | 5473982 | 5474055 | + | May 2004 | Exon1 |
| HB-205 | MT1A | 55229471 | 55229550 | + | May 2004 | Promoter |
| HB-212 | PAX8 | 113709183 | 113709259 | + | May 2004 | Exon 9 |
| HB-043 | DIRAS3 | 68228349 | 68228434 | − | May 2004 | Promoter (in Exon3) |
| HB-199 | PLAGL1 | 1443711135 | 144371211 | + | May 2004 | Exon1 |
| HB-174 | SFN | 26874056 | 26874136 | + | May 2004 | Exon1 |
| HB-289 | SAT2CHRM1 | no perfect | no perfect | | May 2004 | N/A |
| | | match | match | | | |
| HB-493 | MEST | 129919339 | 129919425 | + | March 2006 | exon1/intron1 |
| HB-071 | RNR1 | N/A | N/A | | May 2004 | Promoter |
| HB-076 | ICAM1 | 10242630 | 10242709 | + | May 2004 | Promoter |
| HB-223 | CYP27B1 | 56446731 | 56446808 | − | May 2004 | Exon1 |

| | | | | | | |
|---|---|---|---|---|---|---|
| | | 500 (approx. ± | | Estimated CpG Island | Location of | Location of |
| | | 250) bp sequence | CpG | Length (GenBank) | CpG Island | CpG Island |
| Reaction | HUGO Gene | comprising amplicon | Island | (SEQ ID NO:) | Start (GenBank | End (GenBank |
| Number | Nomenclature | (Genbank sequence) | yes/no | (>0.6 CpG:GpC) | numbering) | numbering) |

| | | | | | | | |
|---|---|---|---|---|---|---|---|
| HB-144 | HRAS | 155726-156225 | (Yes) | 3354 | (SEQ ID NO: 63) | 156171 | 159524 |
| HB-251 | NTF3 | 7301-7800 | Yes | 609 | (SEQ ID NO: 2) | 7246 | 7854 |
| HB-205 | MT1A | 18201-18700 | Yes | 1209 | (SEQ ID NO: 4) | 17842 | 19050 |
| HB-212 | PAX8 | 72426-72925 | Yes | 1250 | (SEQ ID NO: 1) | 73859 | 72610 |
| HB-043 | DIRAS3 | 1751-2250 | Yes | 552 | (SEQ ID NO: 3) | 1804 | 2355 |
| HB-199 | PLAGL1 | 52751-53250 | Yes | 1478 | (SEQ ID NO: 7) | 53667 | 52190 |
| HB-174 | SFN | 8637-9136 | Yes | 661 | (SEQ ID NO: 6) | 8684 | 9344 |
| HB-289 | SAT2CHRM1 | 851-1350 | (Yes) | (500 | (SEQ ID NO: 9)) | N/A | N/A |
| HB-493 | MEST | | Yes | 2799 | (SEQ ID NO: 5) | 4293 | 7091 |
| HB-071 | RNR1 | 1-500 | yes | 850 | (SEQ ID NO: 10) | 1 | 850 |
| HB-076 | ICAM1 | 85376-85875 | Yes | 2038 | (SEQ ID NO: 12) | 84047 | 86084 |
| HB-223 | CYP27B1 | 1501-2000 | yes | 747 | (SEQ ID NO: 11) | 1345 | 2091 |

| Reaction | HUGO Gene | Amplicon Start relative | Reaction | Bisulfite Conversion: |
|---|---|---|---|---|
| Number | Nomenclature | to CGI start | Type | Top/Bottom Strand |
| HB-144 | HRAS | N/A | Methylated | Bottom |
| HB-251 | NTF3 | 257 | Methylated | Top |
| HB-205 | MT1A | 333 | Methylated | Top |
| HB-212 | PAX8 | 1151 | Methylated | Top |
| HB-043 | DIRAS3 | 149 | Methylated | Top |
| HB-199 | PLAGL1 | 622 | Methylated | Top |
| HB-174 | SFN | 116 | Methylated | Top |
| HB-289 | SAT2CHRM1 | N/A | Methylated | Top |
| HB-493 | MEST | 1764 | Methylated | Top |
| HB-071 | RNR1 | 219 | Methylated | Top |
| HB-076 | ICAM1 | 1685 | Methylated | Top |
| HB-223 | CYP27B1 | 383 | Methylated | Top |

Statistical cluster analysis of METHYLIGHT™ data. Hierarchical cluster analysis of 36 loci was performed, using correlation to measure the distance between any two loci and Ward's method of linkage [42]. SASH1 was omitted from the cluster analysis because only a single sample showed positive methylation. The 65 study samples were ordered from left to right by increasing semen concentration.

Display of ILLUMINA™ data. ILLUMINA™ data were displayed graphically in FIG. 3 with results for study samples ordered left to right in columns by sperm concentration. Rows corresponding to each of the 1,421 sequences were divided into three tertiles of median β-value among buffy coat DNA samples (I, II, III), then sorted within tertile by median β-value among all sperm DNA samples. Box 1 contains all sequences tertile I with median β-value among sperm DNA samples >0.5; box 2 contains all sequences within tertile III with median β-value among sperm DNA samples <0.1. Maternal or paternal imprinting status of each locus was scored according to the categorization of R. Jirtle [43]. All sequences specific to genes imprinted in humans were individually reviewed to determine whether they have been reported as belonging to a DMR for which parent of origin marks are maintained by DNA methylation [44-66]. Sequences meeting these criteria were scored as maternally imprinted (MI) or paternally imprinted (PI) with an indicator set for each on FIG. 3.

### Results

Standard semen analysis was conducted on samples collected by 69 men during clinical evaluation of couples with infertility. Among the 69 samples, semen volume ranged from 0.5 to 7.8 ml; total count 0 to 864 million sperm; total motile count 0 to 396.3 million sperm; and percentage normal sperm forms 0 to 26%. Four samples were found to be azoospermic and excluded from subsequent analysis of DNA methylation.

Applicants evaluated 294 METHYLIGHT™ reactions for the presence of methylation in sperm DNA from an anonymous semen sample obtained from a sperm bank. Primers and probes were as previously published (see Table S1 (Sections A-B), found at doi:10.1371/journal.pone.0001289.s001 (0.10 MB PDF); incorporated by reference herein in its entirety; Primers, probes and reaction IDs for 294 MethyLight Assays: Group A, used in screening procedure and analysis of 65 study samples; Group B, used only in screening procedure; and Group C, new assays designed to DMRs of maternally imprinted genes and used only in analysis of 65 study samples.

The 35 selected reactions of Table S1A were used to assay sperm DNA from 65 study samples.

At many of the 35 sequences methylation levels were elevated in DNA from poor quality sperm. For example, striking associations with each of sperm concentration, motility and morphology were observed for five sequences: HRAS, NTF3, MT1A, PAX8 and PLAGL1 (FIG. 1).

PLAGL1 is maternally imprinted. Our METHYLIGHT™ assay for this gene interrogates a differentially methylated CpG island [22]. To determine whether other maternally imprinted genes are methylated in abnormal sperm, METHYLIGHT™ was used to interrogate the differentially methylated sequence of DIRAS3. At this sequence greater DNA methylation was also observed in samples with poorer semen parameters (FIG. 1, row 6). These results appeared to conflict with those of Marques et al [20] who reported no association between low sperm count and methylation of a DMR in a third maternally imprinted gene, MEST. We therefore used METHYLIGHT™ to assess the methylation status of a differentially methylated MEST sequence investigated by these authors [20], and found elevated DNA methylation to be significantly associated with poor semen parameters (FIG. 1), in agreement with our PLAGL1 and DIRAS3 results.

After correction for multiple comparisons, estimated associations between results of each of the 37 METHYLIGHT™ assays and sperm concentration were highly significant for HRAS, NTF3, MT1A, PAX8, DIRAS3 and PLAGL1 and marginally significant for SFN, SAT2CHRM1 and MEST (Table 3, FIG. 1).

TABLE 3
Trend p-values for associations between MethyLight
results and semen parameters (see Methods).

| | Parameter of Standard Semen Analysis | | |
|---|---|---|---|
| MethyLight Reaction | Concentration | Motility | Morphology |
| *HRAS.HB.144 | 0.00006 | 0.00001 | 0.06265 |
| *NTF3.HB.251 | 0.00029 | 0.00026 | 0.00464 |
| MT1A.HB.205 | 0.00048 | 0.00026 | 0.00119 |
| *PAX.8.HB.212 | 0.00086 | 0.00405 | 0.05143 |
| *DIRAS3.HB.043 | 0.00109 | 0.00159 | 0.06016 |
| *PLAGL1.HB.199 | 0.00213 | 0.00255 | 0.01951 |
| *SFN.HB.174 | 0.00307 | 0.00804 | 0.79899 |
| *SAT2CHRM1.HB.289 | 0.00448 | 0.00109 | 0.06793 |
| *MEST.HB.493 | 0.00711 | 0.00373 | 0.00359 |
| RNR1.HB.071 | 0.02 | 0.04 | 0.89 |
| CYP27B1 | 0.02 | 0.05 | 0.10 |
| MADH3.HB.053 | 0.09 | 0.15 | 0.35 |
| BDNF.HB.257 | 0.11 | 0.05 | 0.26 |
| PSEN1.HB.263 | 0.16 | 0.27 | 0.81 |
| CGA.HB.237 | 0.23 | 0.34 | 0.93 |
| SERPINB5.HB.208 | 0.23 | 0.64 | 0.80 |
| ICAM1.HB.076 | 0.24 | 0.29 | 0.05 |
| MINT1.HB.161 | 0.24 | 0.60 | 0.34 |
| PTPN6.HB.273 | 0.24 | 0.09 | 0.08 |
| ALU.HB.296 | 0.25 | 0.29 | 0.87 |
| CYP1B1.HB.239 | 0.28 | 0.42 | 0.61 |
| SP23.HB.301 | 0.28 | 0.48 | 0.48 |
| IFNG.HB.311 | 0.33 | 0.22 | 0.93 |
| C9.HB.403 | 0.37 | 0.35 | 0.89 |
| GP2.HB.400 | 0.41 | 0.39 | 0.94 |
| GATA4.HB.325 | 0.45 | 0.20 | 0.12 |
| UIR.HB.189 | 0.48 | 0.47 | 0.70 |
| TFF1.HB.244 | 0.48 | 0.96 | 0.93 |
| LDLR.HB.219 | 0.51 | 0.39 | 0.11 |
| SASH1.HB.085 | 0.51 | 0.15 | 0.15 |
| ABCB1.HB.051 | 0.54 | 0.27 | 0.16 |
| HOXA10.HB.270 | 0.63 | 0.84 | 0.13 |
| MTHFR.HB.058 | 0.70 | 0.38 | 0.43 |
| LINE1.HB.330 | 0.87 | 0.47 | 0.14 |
| LZTS1.HB.200 | 0.90 | 0.95 | 0.73 |
| SMUG1.HB.086 | 0.90 | 0.36 | 0.76 |
| ^{‡}IGF2.HB.345 | 0.91 | 0.71 | 0.11 |

*Belongs to cluster 2 (see FIG. 2).
^{‡}Assay interrogates a non-differentially methylated sequence.
Trends were assessed over the following categories of semen parameters: Concentration (<5, 5-20, >20 × 10⁶ sperm per ml), Morphology (<5%, 5-14%, >14% normal sperm forms), Motility (<10, 10-50, >50 total motile sperm count (×10⁶)).

Applicants then subjected METHYLIGHT™ data from 36 of the assays to unsupervised cluster analysis. (Data for SASH1 were not included, because methylation at this sequence was detected in only one sample.) This analysis identified three distinct clusters of sequences based on DNA methylation profiles in the 65 samples (FIG. 2). Notably, the middle cluster shown in FIG. 2 includes eight of the nine sequences (all except MT1A) individually associated with semen parameters. This middle cluster includes not only three sequences that are differentially methylated on imprinted loci, but also three single copy sequences specific to non-imprinted genes, and a repetitive element, Satellite 2 [23] (reaction named SAT2CHRM1).

Significantly, this surprising result indicates that sperm abnormalities may be associated with a broad epigenetic defect of elevated DNA methylation at numerous sequences of diverse types, rather than a defect of imprinting alone as previously suggested [20].

To learn more about the possible extent of this apparent defect, the ILLUMINA™ platform was used to conduct DNA methylation analysis of 1,421 sequences in autosomal loci. Included in this analysis was: DNA from the anonymous sperm sample used in the METHYLIGHT™ screen (FIG. 3, columns S); two purchased samples of buffy coat DNA allowing for observation of methylation patterns in somatic cells (FIG. 3, columns 1-2), and seven study sperm DNA samples remaining after METHYLIGHT™ analysis (FIGS. 2-3, columns A-G).

Results of ILLUMINA™ analyses appear in FIG. 3. A large number of genes were similarly methylated in both sperm DNA and buffy coat DNA (blue regions on the left bar, I; red regions on the right bar, III), while others tended to be more methylated in DNA isolated from only one of these cell types. Boxes enclose sequences for which we observed particularly strong patterns of cell type-specific methylation. Box 1 identifies 19 sequences with sperm-specific DNA methylation. At these sequences, methylation profiles of all DNA from samples of study sperm (A-G) closely resemble those from the anonymous sperm sample and differ greatly from those of buffy coat DNA. Box 2 identifies 102 sequences with buffy coat-specific DNA methylation. This set is larger in number than the sperm-specific set, as expected, given that sperm DNA is reportedly hypomethylated compared with somatic cell DNA [14]. The buffy coat-specific set comprises 7.2% of the 1,421 sequences including the majority of DMRs associated with imprinted genes that are on the Illumina panel. At many buffy coat-specific sequences, DNA methylation was elevated in study sperm DNA, most notably in sample A that had been isolated from sperm with the lowest concentration among samples A-G. Methylation of sample A DNA is elevated (β>0.1) at 76 of the 102 sequences in box 2, including all 10 that are known DMRs associated with imprinted genes.

Several factors assure us that our observations did not arise from somatic cell contamination of separated sperm samples [21]. Somatic cells are far larger than sperm and readily identified by microscopic evaluation of semen samples. Even if somatic cells are present in the neat ejaculate, the ISOLATE® sperm separation technique is specifically designed to separate spermatozoa from somatic cells and miscellaneous debris [24]. Moreover, although microscopic evaluation of semen samples conducted before sperm separation identified white blood cells in five of the 65 neat semen samples, excluding results on these five samples from statistical analyses had minimal effect on associations between DNA methylation and semen parameters, and DNA from these samples were excluded from ILLUMINA™ assays.

Various semen parameters have been correlated with abnormal DNA methylation (sperm concentration; total normal morphology; motility, volume, viscosity, etc.). According to preferred aspects, three of these semen parameters show the highest correlations with abnormal DNA methylation: sperm concentration; total normal morphology; and motility. FIG. 2, for example, shows that the corresponding MLL reactions are clustered based on sperm concentration.

Particular preferred aspects, therefore, provide marker(s) and marker subsets having utility for determining at least one of abnormal sperm concentration, abnormal morphology, and abnormal motility.

In particular aspects, with respect to (A) abnormal sperm concentration, markers are provided in the following order of statistical significance from left to right, based on the p-value: HRAS, NTF3, MT1A, PAX8, DIRAS3, PLAGL1, SFN, SAT2CHRM1, and MEST. All of these nine markers have p-values well below 0.05, and therefore, all nine are very significant. Additionally provided are two more markers, RNR1 and CYP27B1, both have p-value of 0.02, that are therefore also of utility in this respect.

In particular aspects, with respect to (B) abnormal total motile sperm, markers are provided in the following order of statistical significance from left to right, based on the p-value: HRAS, NTF3, MT1A (NTF3 and MT1A equally significant), SAT2CHRM1, DIRAS3, PLAGL1, MEST, PAX8, & SFN. Again, these have very significant p-values. Additionally provided are three more markers: RNR1 (p-value 0.04) and CYP27B1, BDNF, both with p-value of 0.05, that are therefore also of utility in this respect.

In particular aspects, with respect to (C) abnormal motility, markers are provided in the following order of statistical significance from left to right, based on the p-value: MT1A, MEST, NTF3, PLAGL1. Additionally, PAX8 AND ICAM1 both have p-values of 0.05, and are thus also of utility in this respect.

### Example 2

### Additional Aspects Provide Methods for Screening for Agents that Cause Spermatogenic Deficits, Abnormal Sperm or Abnormal Fertility

### Overview

As stated herein above, this is the first study ever to describe the epigenetic state of abnormal human sperm using an extensive panel of DNA methylation assays. According to additional aspects, Applicants data has provided novel methylation-based markers for abnormal human sperm and/or fertility.

As recognized in the art, transient in vivo chemical exposure at 7-15 days post conception, which includes the analogous stage of murine development [29,30], results in spermatogenic deficits in rats with grossly normal testes [31] but likely associated with elevated methylation of sperm DNA [32].

According to additional aspects, therefore, Applicants' data provides for methods for screening for agents that cause spermatogenic deficits, abnormal sperm or abnormal fertility. In particular aspects, ES-cell derived primordial germ cells are exposed to chemical test agents, followed by CpG methylation analysis as described and provided for herein, to allow for a high-throughput screening assay to test and identify agents that cause spermatogenic deficits, abnormal sperm or abnormal fertility. Culturing of embryonic stem (ES) cells to efficiently provide for primordial germ cells is known in the art. For example, human embryonic stem (ES) cells are propagated on mouse embryo fibroblast feeder cells as described (67). A multistep induction procedure incorporating several previously described protocols can be used to convert ES cells into primordial germ cells at high efficiency. For example, ES cells are treated with bone morphogenetic protein-2 for a brief 24 period in combination with activin and FGF-2 in chemically defined medium. After 24 hours the BMP-2 is removed and retinoic acid is added. As will be appreciated in the art, a range of doses of each factor may be employed in a matrix design over a variable time course to optimize the yield of c-kit positive/placental alkaline phosphatase positive cells. These cells are isolated by flow cytometry and subjected to Q-RTPCR to analyze for the presence of primordial germ cell and gonocyte specific genes such as VASA. According to particular aspects, up to 10% of the treated cells are vasa positive following optimal treatment. Primordial germ cells and gonocytes may also be isolated from embryonic and fetal gonads by the use of c-kit and placental alkaline phosphatase in combination with flow cytometry, following collagenase and Tryple Express™ digestion of the tissue.

Particular aspects, therefore, provide methods for screening for agents that cause spermatogenic deficits, abnormal sperm or abnormal fertility comprising: obtaining human ES-cell derived primordial germ cells; contacting the germ cells or descendants thereof, with at least one test agent; culturing the contacted germ cells or the descendants thereof under conditions suitable for germ cell proliferation or development; obtaining a sample of genomic DNA from the contacted cultured germ cells or the descendants thereof; determining, using the genomic DNA of the sample, the methylation status of at least one CpG dinucleotide sequence of at least one gene sequence selected from the group consisting of HRAS, NTF3, MT1A, PAX8, DIRAS3, PLAGL1, SFN, SAT2CHRM1, MEST, RNR1, CYP27B1 and ICAM1; and identifying, based on the methylation status of the at least one CpG sequence, at least one test agent that causes at least one of spermatogenic deficits, abnormal sperm, and abnormal fertility. In certain embodiments, the determined methylation status of the at least one CpG sequence is hypermethylation. In preferred embodiments, the at least one gene sequence is selected from the group consisting of HRAS SEQ ID NOS:63 and 20, NTF3 SEQ ID NOS:2 and 14, MT1A SEQ ID NOS:4 and 16, PAX8 SEQ ID NOS:1 and 13, DIRAS3 SEQ ID NOS:3 and 15, PLAGL1 SEQ ID NOS:7 and 19, SFN SEQ ID NOS:6 and 18, SAT2CHRM1 SEQ ID NOS:9 and 21, MEST SEQ ID NOS:5 and 17, RNR1 SEQ ID NOS:10 and 22, CYP27B1 SEQ ID NOS:11 and 23 and ICAM1 SEQ ID NOS:12 and 24.

### REFERENCES CITED AND INCORPORATED HEREIN BY REFERENCE

1. Sokol R Z (1997) male factor in male infertility. In: Lobo R. M D, Paulson R., editor. Infertility, Contraception, and Reproductive Endocrinology. Madden, M A: Blackwell Science, Inc, pp. 547-566.
2. Thonneau P, Marchand S, Tallec A, Ferial M L, Ducot B et al. (1991) Incidence and main causes of infertility in a resident population (1,850,000) of three French regions (1988-1989). Hum Reprod 6(6): 811-816.
3. Maduro M R, Lo K C, Chuang W W, Lamb D J (2003) Genes and male infertility: what can go wrong? J Androl 24(4): 485-493.
4. McElreavey K, Krausz C, Bishop C E (2000) The human Y chromosome and male infertility. Results Probl Cell Differ 28: 211-232.
5. Sharlip I D, Jarow J P, Belker A M, Lipshultz L I, Sigman M et al. (2002) Best practice policies for male infertility. Fertil Steril 77(5): 873-882.
6. Rousseaux S, Caron C, Govin J, Lestrat C, Faure A K et al. (2005) Establishment of male-specific epigenetic information. Gene 345(2): 139-153.
7. Emery B R, Carrell D T (2006) The effect of epigenetic sperm abnormalities on early embryogenesis. Asian J Androl 8(2): 131-142.
8. Li E (2002) Chromatin modification and epigenetic reprogramming in mammalian development. Nat Rev Genet 3(9): 662-673.
9. Saitou M, Barton S C, Surani M A (2002) A molecular programme for the specification of germ cell fate in mice. Nature 418(6895): 293-300.
10. Santos F, Dean W (2004) Epigenetic reprogramming during early development in mammals. Reproduction 127(6): 643-651.
11. Biermann K, Steger K (2007) Eipgenetics in Male Germ Cells. J Androl.
12. Ariel M, Cedar H, McCarrey J (1994) Developmental changes in methylation of spermatogenesis-specific genes include reprogramming in the epididymis. Nat Genet 7(1): 59-63.
13. Trasler J M (1998) Origin and roles of genomic methylation patterns in male germ cells. Semin Cell Dev Biol 9(4): 467-474.
14. Oakes C C, La Salle S, Smiraglia D J, Robaire B, Trasler J M (2007) A unique configuration of genome-wide DNA methylation patterns in the testis. Proc Natl Acad Sci USA 104(1): 228-233.
15. Bestor T H, Bourc'his D (2004) Transposon silencing and imprint establishment in mammalian germ cells. Cold Spring Harb Symp Quant Biol 69: 381-387.
16. Schaefer C B, Ooi S K, Bestor T H, Bourc'his D (2007) Epigenetic decisions in mammalian germ cells. Science 316(5823): 398-399.
17. Flanagan J M, Popendikyte V, Pozdniakovaite N, Sobolev M, Assadzadeh A et al. (2006) Intra- and interindividual epigenetic variation in human germ cells. Am J Hum Genet 79(1): 67-84.
18. Benchaib M, Braun V, Ressnikof D, Lornage J, Durand P et al. (2005) Influence of global sperm DNA methylation on IVF results. Hum Reprod 20(3): 768-773.
19. Hartmann S, Bergmann M, Bohle R M, Weidner W, Steger K (2006) Genetic imprinting during impaired spermatogenesis. Mol Hum Reprod 12(6): 407-411.
20. Marques C J, Carvalho F, Sousa M, Barros A (2004) Genomic imprinting in disruptive spermatogenesis. Lancet 363(9422): 1700-1702.
21. Manning M, Lissens W, Liebaers I, Van Steirteghem A, Weidner W (2001) Imprinting analysis in spermatozoa prepared for intracytoplasmic sperm injection (ICSI). Int J Androl 24(2): 87-94.
22. Varrault A, Bilanges B, Mackay D J, Basyuk E, Ahr B et al. (2001) Characterization of the methylation-sensitive promoter of the imprinted ZAC gene supports its role in transient neonatal diabetes mellitus. J Biol Chem. pp. 18653-18656.
23. Weisenberger D J, Siegmund K D, Campan M, Young J, Long T I et al. (2006) CpG island methylator phenotype underlies sporadic microsatellite instability and is tightly associated with BRAF mutation in colorectal cancer. Nat Genet 38(7): 787-793.
24. Dale B, Elder, K. (1997) In vitro fertilization. New York: Cambridge University Press. 187 p.
25. Hayashi K, de Sousa Lopes S M, Surani M A (2007) Germ cell specification in mice. Science 316(5823): 394-396.
26. Yabuta Y, Kurimoto K, Ohinata Y, Seki Y, Saitou M (2006) Gene expression dynamics during germline specification in mice identified by quantitative single-cell gene expression profiling. Biol Reprod 75(5): 705-716.
27. Surani M A, Hayashi K, Hajkova P (2007) Genetic and epigenetic regulators of pluripotency. Cell 128(4): 747-762.
28. Kerjean A, Dupont J M, Vasseur C, Le Tessier D, Cuisset L et al. (2000) Establishment of the paternal methylation imprint of the human H19 and MEST/PEG1 genes during spermatogenesis. Hum Mol Genet 9(14): 2183-2187.
29. Lee J, Inoue K, Ono R, Ogonuki N, Kohda T et al. (2002) Erasing genomic imprinting memory in mouse clone embryos produced from day 11.5 primordial germ cells. Development 129(8): 1807-1817.
30. Hajkova P, Erhardt S, Lane N, Haaf T, El-Maarri O et al. (2002) Epigenetic reprogramming in mouse primordial germ cells. Mech Dev 117(1-2): 15-23.
31. Cupp A S, Uzumcu M, Suzuki H, Dirks K, Phillips B et al. (2003) Effect of transient embryonic in vivo exposure to the endocrine disruptor methoxychlor on embryonic and postnatal testis development. J Androl 24(5): 736-745.
32. Chang H S, Anway M D, Rekow S S, Skinner M K (2006) Transgenerational epigenetic imprinting of the male germline by endocrine disruptor exposure during gonadal sex determination. Endocrinology 147(12): 5524-5541.
33. Acacio B D, Gottfried T, Israel R, Sokol R Z (2000) Evaluation of a large cohort of men presenting for a screening semen analysis. Fertil Steril 73(3): 595-597.
34. World Health Organization Laboratory Manual for Human Semen and Sperm Cervical Mucus Interaction (1999).
35. Guzick D S, Overstreet J W, Factor-Litvak P, Brazil C K, Nakajima S T et al. (2001) Sperm morphology, motility, and concentration in fertile and infertile men. N Engl J Med 345(19): 1388-1393.
36. www.irvinesci.com (2006).
37. Laird P W, Zijderveld A, Linders K, Rudnicki M A, Jaenisch R et al. (1991) Simplified mammalian DNA isolation procedure. Nucleic Acids Res 19(15): 4293.
38. Bibikova M, Lin Z, Zhou L, Chudin E, Garcia E W et al. (2006) High-throughput DNA methylation profiling using universal bead arrays. Genome Res 16(3): 383-393.
39. www.illumina.com/pages.ilmn?ID=193 (2007).
40. http://www.illumina.com (2007).
41. Benjamini y, Hochberg y (1995) Controlling the false discovery rate: a practical and powerful approach to multiple testing. R Statist Soc B 57(1): 289-300.
42. Kaufman L, Rousseeuw P J (1990) Finding Groups in Data: An introduction to cluster analysis. Wiley Series in Probability and Mathematical Statistics. New York: John Wiley & Sons, Inc.
43. www.geneimprint.com (2006).
44. Astuti D, Latif F, Wagner K, Gentle D, Cooper W N et al. (2005) Epigenetic alteration at the DLK1-GTL2 imprinted domain in human neoplasia: analysis of neuroblastoma, phaeochromocytoma and Wilms' tumour. Br J Cancer 92(8): 1574-1580.
45. Bastepe M, Frohlich L F, Hendy G N, Indridason O S, Josse R G et al. (2003) Autosomal dominant pseudohypoparathyroidism type Ib is associated with a heterozygous microdeletion that likely disrupts a putative imprinting control element of GNAS. J Clin Invest 112(8): 1255-1263.
46. Bastepe M, Frohlich L F, Linglart A, Abu-Zahra H S, Tojo K et al. (2005) Deletion of the NESP55 differentially methylated region causes loss of maternal GNAS imprints and pseudohypoparathyroidism type Ib. Nat Genet 37(1): 25-27.
47. Bell A C, Felsenfeld G (2000) Methylation of a CTCF-dependent boundary controls imprinted expression of the lgf2 gene. Nature 405(6785): 482-485.
48. de la Puente A, Hall J, Wu Y Z, Leone G, Peters J et al. (2002) Structural characterization of Rasgrf1 and a novel linked imprinted locus. Gene 291(1-2): 287-297.
49. Gaston V, Le Bouc Y, Soupre V, Burglen L, Donadieu J et al. (2001) Analysis of the methylation status of the KCNQ1OT and H19 genes in leukocyte DNA for the diagnosis and prognosis of Beckwith-Wiedemann syndrome. Eur J Hum Genet 9(6): 409-418.
50. Higashimoto K, Soejima H, Saito T, Okumura K, Mukai T (2006) Imprinting disruption of the CDKN1C/KCNQ1OT1 domain: the molecular mechanisms causing Beckwith-Wiedemann syndrome and cancer. Cytogenet Genome Res 113(1-4): 306-312.
51. Jie X, Lang C, Jian Q, Chaoqun L, Dehua Y et al. (2007) Androgen activates PEG10 to promote carcinogenesis in hepatic cancer cells. Oncogene.
52. Liu J, Nealon J G, Weinstein L S (2005) Distinct patterns of abnormal GNAS imprinting in familial and sporadic pseudohypoparathyroidism type IB. Hum Mol Genet 14(1): 95-102.
53. Murphy S K, Wylie A A, Jirtle R L (2001) Imprinting of PEG3, the human homologue of a mouse gene involved in nurturing behavior. Genomics 71(1): 110-117.
54. Runte M, Huttenhofer A, Gross S, Kiefmann M, Horsthemke B et al. (2001) The IC-SNURF-SNRPN transcript serves as a host for multiple small nucleolar RNA species and as an antisense RNA for UBE3A. Hum Mol Genet 10(23): 2687-2700.
55. Runte M, Kroisel P M, Gillessen-Kaesbach G, Varon R, Horn D et al. (2004) SNURF-SNRPN and UBE3A transcript levels in patients with Angelman syndrome. Hum Genet 114(6): 553-561.
56. Sutcliffe J S, Nakao M, Christian S, Orstavik K H, Tommerup N et al. (1994) Deletions of a differentially methylated CpG island at the SNRPN gene define a putative imprinting control region. Nat Genet 8(1): 52-58.
57. Suzuki S, Ono R, Narita T, Pask A J, Shaw G et al. (2007) Retrotransposon silencing by DNA methylation can drive mammalian genomic imprinting. PLoS Genet 3(4): e55.
58. Vu T H, Li T, Nguyen D, Nguyen B T, Yao X M et al. (2000) Symmetric and asymmetric DNA methylation in the human IGF2-H19 imprinted region. Genomics 64(2): 132-143.
59. Cui H, Onyango P, Brandenburg S, Wu Y, Hsieh C L et al. (2002) Loss of imprinting in colorectal cancer linked to hypomethylation of H19 and IGF2. Cancer Res 62(22): 6442-6446.
60. Hancock A L, Brown K W, Moorwood K, Moon H, Holmgren C et al. (2007) A CTCF-binding silencer regulates the imprinted genes AWT1 and WT1-AS and exhibits sequential epigenetic defects during Wilms' tumourigenesis. Hum Mol Genet 16(3): 343-354.
61. Kim J D, Hinz A K, Choo J H, Stubbs L, Kim J (2007) YY1 as a controlling factor for the Peg3 and Gnas imprinted domains. Genomics 89(2): 262-269.
62. Lin S P, Youngson N, Takada S, Seitz H, Reik W et al. (2003) Asymmetric regulation of imprinting on the maternal and paternal chromosomes at the Dlk1-Gtl2 imprinted cluster on mouse chromosome 12. Nat Genet 35(1): 97-102.
63. Murrell A, Heeson S, Reik W (2004) Interaction between differentially methylated regions partitions the imprinted genes lgf2 and H19 into parent-specific chromatin loops. Nat Genet 36(8): 889-893.
64. Ono R, Kobayashi S, Wagatsuma H, Aisaka K, Kohda T et al. (2001) A retrotransposon-derived gene, PEG10, is a novel imprinted gene located on human chromosome 7q21. Genomics 73(2): 232-237.
65. Sullivan M J, Taniguchi T, Jhee A, Kerr N, Reeve A E (1999) Relaxation of IGF2 imprinting in Wilms tumours associated with specific changes in IGF2 methylation. Oncogene 18(52): 7527-7534.
66. Yun J, Park C W, Lee Y J, Chung J H (2003) Allele-specific methylation at the promoter-associated CpG island of mouse Copg2. Mamm Genome 14(6): 376-382.
67. Reubinoff B E, Pera M F, Fong C Y, Trounson A, Bongso A. Embryonic stem cell lines from human blastocysts: somatic differentiation in vitro. Nat Biotechnol 18 399-404, 2000.

1. A method for determining or diagnosing abnormal sperm or fertility, comprising:
obtaining a sample of human sperm DNA from a test subject;
determining, using the genomic DNA of the sample, the methylation status of at least one CpG dinucleotide sequence of at least one gene sequence selected from the group consisting of HRAS, NTF3, MT1A, PAX8, DIRAS3, PLAGL1, SFN, SAT2CHRM1, MEST, RNR1, CYP27B1 and ICAM1; and
determining, based on the methylation status of the at least one CpG sequence, the presence or diagnosis of abnormal sperm or fertility with respect to the test subject.

2. The method of claim 1, wherein the determined methylation status of the at least one CpG sequence is hypermethylation.

3. The method of claim 1, wherein determining the methylation status of at least one CpG dinucleotide sequence comprises treating the genomic DNA, or a fragment thereof, with one or more reagents to convert 5-position unmethylated cytosine bases to uracil or to another base that is detectably dissimilar to cytosine in terms of hybridization properties.

4. The method of claim 3, wherein treating comprises use of bisulfite treatment of the DNA.

5. The method of claim 1, wherein the at least one gene sequence is selected from the group consisting of HRAS SEQ ID NOS:63 and 20, NTF3 SEQ ID NOS:2 and 14, MT1A SEQ ID NOS:4 and 16, PAX8 SEQ ID NOS:1 and 13, DIRAS3 SEQ ID NOS:3 and 15, PLAGL1 SEQ ID NOS:7 and 19, SFN SEQ ID NOS:6 and 18, SAT2CHRM1 SEQ ID NOS:9 and 21, MEST SEQ ID NOS:5 and 17, RNR1 SEQ ID NOS:10 and 22, CYP27B1 SEQ ID NOS:11 and 23 and ICAM1 SEQ ID NOS:12 and 24.

6. The method of claim 1, wherein abnormal sperm comprises at least one of abnormal sperm concentration, abnormal motility, abnormal total normal morphology, abnormal volume, and abnormal viscosity.

7. The method of claim 6, wherein abnormal sperm comprises at least one of abnormal sperm concentration, abnormal motility, and abnormal total normal morphology.

8. The method of claim 7, comprising determining, using the genomic DNA of the sample, the methylation status of at least one CpG dinucleotide sequence of at least one gene sequence selected from the group consisting of HRAS, NTF3, MT1A, PAX8 and PLAGL1.

9. The method of claim 8, wherein the at least one gene sequence is selected from the group consisting of HRAS SEQ ID NOS:63 and 20, NTF3 SEQ ID NOS:2 and 14, MT1A SEQ ID NOS:4 and 16, PAX8 SEQ ID NOS:1 and 13, and PLAGL1 SEQ ID NOS:7 and 19.

10. A method for determining or diagnosing abnormal sperm or fertility, comprising:
obtaining a sample of human sperm DNA from a test subject;
determining, using the genomic DNA of the sample, the methylation status of at least one CpG dinucleotide sequence of at least one gene sequence from each of a repetitive DNA element sequence group, a maternally imprinted gene sequence group, and a non-imprinted gene sequence group; and
determining, based on the methylation status of the at least one CpG sequence from each of the groups, the presence or diagnosis of abnormal sperm or fertility with respect to the test subject.

11. The method of claim 10, wherein the determined methylation status of the at least one CpG sequence is hypermethylation.

12. The method of claim 10, wherein determining the methylation status of at least one CpG dinucleotide sequence comprises treating the genomic DNA, or a fragment thereof, with one or more reagents to convert 5-position unmethylated cytosine bases to uracil or to another base that is detectably dissimilar to cytosine in terms of hybridization properties.

13. The method of claim 12, wherein treating comprises use of bisulfite treatment of the DNA.

14. The method of claim 10, wherein the at least one gene sequence from a repetitive element group comprises at least one selected from the group consisting of SAT2CHRM1 SEQ ID NOS:9 and 21.

15. The method of claim 10, wherein the at least one gene sequence from a maternally imprinted gene group comprises at least one selected from the group consisting of PLAGL1 SEQ ID NOS:7 and 19, MEST SEQ ID NOS:5 and 17, and DIRAS3 SEQ ID NOS:3 and 15.

16. The method of claim 10, wherein the at least one gene sequence from a non-imprinted gene group comprises at least one selected from the group consisting of HRAS SEQ ID NOS:63 and 20, NTF3 SEQ ID NOS:2 and 14, MT1A SEQ ID NOS:4 and 16, PAX8 SEQ ID NOS:1 and 13, SFN SEQ ID NOS:6 and 18, RNR1 SEQ ID NOS:10 and 22, CYP27B1 SEQ ID NOS:11 and 23 and ICAM1 SEQ ID NOS:12 and 24.

17. A method for screening for agents that cause spermatogenic deficits, abnormal sperm or abnormal fertility comprising:
obtaining human ES-cell derived primordial germ cells;
contacting the germ cells or descendants thereof, with at least one test agent;
culturing the contacted germ cells or the descendants thereof under conditions suitable for germ cell proliferation or development;
obtaining a sample of genomic DNA from the contacted cultured germ cells or the descendants thereof;
determining, using the genomic DNA of the sample, the methylation status of at least one CpG dinucleotide sequence of at least one gene sequence selected from the group consisting of HRAS, NTF3, MT1A, PAX8, DIRAS3, PLAGL1, SFN, SAT2CHRM1, MEST, RNR1, CYP27B1 and ICAM1; and
identifying, based on the methylation status of the at least one CpG sequence, at least one test agent that causes at least one of spermatogenic deficits, abnormal sperm, and abnormal fertility.

18. The method of claim 17, wherein the determined methylation status of the at least one CpG sequence is hypermethylation.

19. The method of claim 17, wherein determining the methylation status of at least one CpG dinucleotide sequence comprises treating the genomic DNA, or a fragment thereof, with one or more reagents to convert 5-position unmethylated cytosine bases to uracil or to another base that is detectably dissimilar to cytosine in terms of hybridization properties.

20. The method of claim 19, wherein treating comprises use of bisulfite treatment of the DNA.

21. The method of claim 17, wherein the at least one gene sequence is selected from the group consisting of HRAS SEQ ID NOS:63 and 20, NTF3 SEQ ID NOS:2 and 14, MT1A SEQ ID NOS:4 and 16, PAX8 SEQ ID NOS:1 and 13, DIRAS3 SEQ ID NOS:3 and 15, PLAGL1 SEQ ID NOS:7 and 19, SFN SEQ ID NOS:6 and 18, SAT2CHRM1 SEQ ID NOS:9 and 21, MEST SEQ ID NOS:5 and 17, RNR1 SEQ ID NOS:10 and 22, CYP27B1 SEQ ID NOS:11 and 23 and ICAM1 SEQ ID NOS:12 and 24.
